(19) United States
(12) Patent Application Publication
(10) Pub. No.: US 2009/0246155 A1
(43) Pub. Date: Oct. 1, 2009

(54) Compositions and methods for personal care

(75) Inventors: Steven P. Bitler, Menlo Park, CA (US); Qiang Zheng, Palo Alto, CA (US); David Taft, Atherton, CA (US)

Correspondence Address: BELL & ASSOCIATES, 58 West Portal Avenue No. 121, SAN FRANCISCO, CA, 94127, US

(73) Assignee: Landec Corporation

(21) Appl. No.: 12/455,628

(22) Filed: Jun. 4, 2009

Related U.S. Application Data
(63) Continuation-in-part of application No. 11/999,415, filed on Dec. 4, 2007.
(60) Provisional application No. 60/873,234, filed on Dec. 5, 2006.
(60) Provisional application No. 61/131,123, filed on Jun. 4, 2008.
(60) Provisional application No. 61/131,716, filed on Jun. 10, 2008.

(51) Int. Cl. A61Q 17/04; A61K 8/00; A61Q 15/00; A61Q 1/06; A61Q 1/00; A61Q 5/00; A61Q 3/00; C11D 3/00

(52) U.S. Cl. 424/59; 424/65; 424/64; 424/63; 424/70.1; 424/61; 510/108

(57) ABSTRACT

Systems and methods for delivering personal care materials. Crystalline polymeric systems, referred to as CYC carriers, are associated with the cosmetic materials, through chemical bonding or through physical association. The crystallinity of the CYC carriers results from the presence of crystallizable side chains, for example long chain n-alkyl moieties, which results in relatively low and sharp melting temperatures. One class of CYC carriers, referred to as CYSC polymers, have a majority of the crystallizable side chains pendant from the polymer backbone. Another class of CYC carriers, referred to as ECC polymers, have a majority of the crystallizable side chains attached to terminal units of the polymer backbone. The ECC polymers can for example be obtained by modification of PLGA polymers. The CYC carriers in another class are non-polymeric. Some CYC carriers, referred to as CYC assemblies, have enhanced crystallinity as a result of the physical association of crystallizable moieties which are present in different types of molecule, for example between a polymer containing crystallizable moieties and a monomer containing crystallizable moieties. Preferably the CYC carrier is bioerodable.

### CROSS-REFERENCE TO RELATED APPLICATIONS

This application is a continuation-in-part of U.S. application Ser. No. 11/999,415, filed Dec. 4, 2007 (Docket 001 US), which claims priority from and the benefit of U.S. provisional Application No. 60/873,234, filed Dec. 5, 2006 (Docket 001 PRV). This application also claims priority from, and the benefit of,
(1) U.S. provisional application 61/131,123, filed Jun. 4, 2008 (Docket 004 PRV) and
(2) U.S. provisional application 61/131,716, filed Jun. 10, 2008 (Docket 004B PRV).
   This application is also related to
(1) International Patent Application PCT/US 2007/024909 (Docket 002 PCT), which was (i) filed Dec. 4, 2007, claiming priority from U.S. Provisional Application 60/873,234 filed Dec. 5, 2006, and (ii) published as WO/2008/070118 on Jun. 12, 2008,
(2) International Patent Application PCT/US 2007/025032 (Docket 002 PCT), which was (i) filed Dec. 4, 2007, claiming priority from U.S. Provisional Application 60/873,234 filed Dec. 5, 2006, and (ii) published as WO/2008/070165 on Jun. 12, 2008,
(3) U.S. application Ser. No. 12/284,755, filed Sep. 25, 2008 (Docket 005 US), which is a continuation of U.S. application Ser. No. 11/999,415, and which claims priority from U.S. provisional Application Nos. 61/005,400, filed Dec. 4, 2007 (Docket 003 PRV), 61/131,123, filed Jun. 4, 2008 (Docket 004 PRV), and 61/131,716, filed Jun. 10, 2008 (Docket 004B PRV),
(4) U.S. application Ser. No. 12/287,520 filed Oct. 10, 2008 (Docket 006 US), which claims priority from U.S. provisional Application Nos. 61/005,400, filed Dec. 4, 2007 (Docket 003 PRV), 61/131,123, filed Jun. 4, 2008 (Docket 004 PRV), and 61/131,716, filed Jun. 10, 2008 (Docket 004B PRV)
(5) U.S. application Ser. No. 12/315,876, filed Dec. 4, 2008 (Docket 008 US), which claims priority from U.S. provisional Application Nos. 61/005,400, filed Dec. 4, 2007 (Docket 003 PRV), 61/131,123, filed Jun. 4, 2008 (Docket 004 PRV), and 61/131,716, filed Jun. 10, 2008 (Docket 004B PRV), and
(6) US Application No. (not yet known) filed contemporaneously with this application (Docket 011 US), and entitled Systems and Methods for Delivery of Materials In Agriculture and Aquaculture, and which claims priority from U.S. provisional Application Nos. 61/131,123, filed Jun. 4, 2008 (Docket 004 PRV), and 61/131,716, filed Jun. 10, 2008 (Docket 004B PRV).
   The entire disclosure of each of the applications and publications identified above is incorporated by reference herein for all purposes.

U.S. application Ser. Nos. 11/999,415 and 12/315,876, and the other patent applications noted above, disclose the use of certain materials (referred to in U.S. application Ser. No. 12/315,876 as “CYC carriers”) for the delivery of drugs, the term “drug” being defined as “a material which is biologically active in a human being or other mammal, locally and/or systemically”, including any chemical moiety that can be used for diagnosis or in a diagnostic test.

### FIELD OF THE INVENTION

This invention relates to compositions and methods for personal care.

### BACKGROUND

There are many known polymeric systems for use in personal care.

### SUMMARY OF THE INVENTION

This invention provides novel compositions and methods which are useful for personal care (including cosmetic surgery). The invention enables the delivery of materials to personal care sites by associating the materials with (i) certain polymers which comprise repeating units containing crystalline moieties and/or terminal units comprising crystalline moieties (those polymers being referred to herein as CYC polymers), and/or (ii) certain non-polymeric compounds which contain crystalline moieties (those compounds being referred to herein as CYC compounds), and/or (iii) certain “self-assemblies” which contain crystalline moieties (those assemblies being referred to herein as CYC assemblies”). The CYC polymers, CYC compounds and CYC assemblies are collectively referred to herein as “CYC carriers”.

Definitions of CYC polymers, CYC compounds, CYC assemblies and SSP polymers (a limited class of CYC polymers) are given below. Where the disclosure below refers to one of the CYC carriers, a component of one of the CYC carriers, a characteristic of one of CYC carriers, or to a composition or method making use of one of the CYC carriers, that disclosure is also applicable to other CYC carriers, unless the context makes this impossible. The materials that can be delivered in accordance with the invention (which are referred to herein as “cosmetic materials”) are materials which are not “drugs” as defined above and which, at the time of delivery or after delivery to the human body, change (generally improve or preserve) the appearance of, and/or hygiene of, and/or smell associated with, the human body. The cosmetic materials can be delivered in combination with materials which are “drugs” as defined above.

The personal care sites to which the materials can be delivered (which are also referred to herein as “target sites”) can be any part of the human body (including, but not limited to, skin, hair and nails).

The term “CYC polymer” is defined herein as a polymer which:

(A) comprises polymeric molecules having a polymer backbone and comprising at least one moiety which

(i) has the formula -b-Cy, and

(ii) either
(A) forms part of a repeating unit of the polymer backbone, the repeating unit having formula (1) below

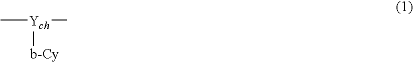

where Y_{ch} is a moiety forming part of the polymer backbone,
b is a bond or moiety which links the Cy moiety to Y_{ch}, and
Cy is a moiety which is associated with other moieties (which may also be Cy moieties) to provide the CYC polymer with crystallinity;
or
(B) forms part of a terminal unit of the polymer backbone, the terminal unit having formula (2) below

-Yₜₑᵣₘ-b-Cy (2)
where Yₜₑᵣₘ is a moiety at the end of the backbone, and b and Cy are as defined in formula (1); and
   (B) has a crystalline melting temperature (hereinafter abbreviated to Tp) of at least 0° C. and a heat of fusion (hereinafter abbreviated to ΔH) of at least 3 J/g which result from association of the Cy moieties. In this definition, and throughout this specification, Tp and ΔH are measured on a differential scanning calorimeter (DSC) as hereinafter described.

In some CYC polymers, the backbone of the polymeric molecules comprises repeating units having formula (3) below

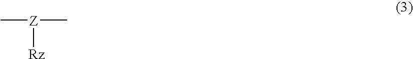

where Z is a moiety forming part of the backbone and Rz represents a moiety which does not comprise a Cy moiety. Many useful CYC polymers of this kind have an amphiphilic character, with the Cy moieties providing hydrophobic characteristics and the Z(Rz) moieties providing hydrophilic characteristics.

The term “CYSC polymer” is used herein to denote a CYC polymer in which at least a majority by weight, preferably at least 90% by weight, particularly substantially all, of the Cy moieties are present in repeating units of formula (1). Thus, a CYSC polymer always contains repeating units of formula (1), and optionally contains terminal units of formula (2) and repeating units of formula (3).

The term “ECC polymer” is used herein to denote a CYC polymer in which at least a majority by weight, preferably at least 90% by weight, particularly substantially all, of the Cy moieties are present in terminal units of formula (2). Thus, an ECC polymer always contains terminal units of formula (2) and repeating units of formula (3), and optionally contains repeating units of formula (1).

The term “CYC compound” is defined herein as a non-polymeric compound which
(A) has the formula

Q(-b-Cy)_{q} (4)
wherein q is least 2, e.g. 3-8,
Q is a moiety having a valence of at least q,
b is a bond or a moiety linking the Cy moiety to the Q moiety, and
Cy is as defined in formula (1), and
(B) has a crystalline melting temperature, Tp, of at least 0° C. and a ΔH of at least 3 J/g which results from association of the Cy moieties.

The term “CYC assembly” is defined herein as an assembly of (i) a polymer which is a CYC polymer as defined above except that the polymer does not necessarily have a Tp of at least 0° C. and a ΔH of at least 4 J/g, and (ii) a compound which contains a Cy moiety and which is intimately mixed with the polymer but is not covalently linked to the polymer, the assembly having a crystalline melting temperature, Tp, of at least 0° C. and a ΔH of at least 3 J/g which results from association of the Cy moieties.

The term SSP polymer is defined herein as a polymer which
(1) has a crystalline melting temperature, Tp, of at least 25° C., e.g. 27-100° C., and a ΔH of at least 5 J/g; and
(2) comprises polymeric molecules having a backbone which comprises
   (a) repeating units which do not contain hydrophilic moieties, and have the formula (1) below,

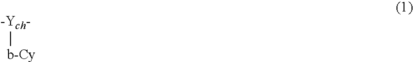

where
   Y_{ch} is a moiety forming part of the backbone,
   b is a bond or a moiety linking the Cy moiety to Y_{ch}, and
   Cy is a moiety is associated with other Cy moieties to provide the SSP polymer with crystallinity;
(b) repeating units which have the formula (2zphil) below,

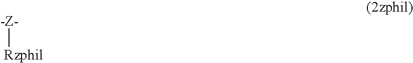

where Z is a moiety forming part of the backbone, and
Rzphil comprises a hydrophilic moiety;
the molar ratio of the units of formula (2zphil) to the units of formula (1) being at least 2.5:1.

The moiety -b-Cy is also referred to in this specification as an -Rc moiety, i.e. Rc is synonymous with b-Cy.

Various aspects of the invention include:—

(1) Personal care compositions comprising (i) a CYC carrier, and (ii) a cosmetic material associated with the CYC carrier. Such compositions are referred to herein as “release compositions”.
(2) Methods of releasing cosmetic materials from compositions as defined in (1).
(3) Methods in which (i) a CYC carrier and (ii) a cosmetic material are used in association to change a target site of a human being, the CYC carrier and the release material being associated with each other before administration or becoming associated with each other during or after administration.
(4) Methods of making a release composition as defined in (1). The composition is often made before the composition is administered, but can be made or modified during administration and/or in situ at the administration site as a result of simultaneous or sequential administration of CYC carrier and the cosmetic material.
(5) Devices for administering (i) a CYC carrier and (ii) a cosmetic material, the CYC carrier and the cosmetic material being (i) in the form of a composition as defined in (1) or (ii) separately administered and becoming associated during or after administration. Such devices are referred to herein as “release devices”.
(6) The use of a release composition or release device as defined above.
(7) Methods of making release devices and release compositions as defined above.

### DETAILED DESCRIPTION OF THE INVENTION

In this specification:—

(1) Reference is made to particular features of the invention (including for example components, ingredients, elements, devices, apparatus, systems, groups, ranges, method steps, test results, etc). It is to be understood that the disclosure of the invention in this specification includes all possible combinations of such particular features. For example, where a particular feature is disclosed in the context of a particular embodiment or claim, that feature can also be used, to the extent appropriate, in the context of other particular embodiments and claims, and in the invention generally.
(2) The singular forms “a”, “an”, and “the” include plural reference unless the context clearly dictates otherwise. Thus, for example, a reference to “a part” includes a plurality of such parts.
(3) The term “comprises” and grammatical equivalents thereof are used to mean that, in addition to the features specifically identified, other features are optionally present. For example a formulation which comprises a CYC carrier and a release material can contain a single CYC carrier and a single release material, or two or more CYC carriers and/or two or more release materials, and optionally contains one or more other ingredients which are not CYC carriers, for example other ingredients as disclosed herein.
(4) The term “consisting essentially of” and grammatical equivalents thereof are used to mean that, in addition to the features specifically identified, other features may be present which do not materially alter the disclosed and/or claimed invention.
(5) The term “at least” followed by a number is used to denote the start of a range beginning with that number (which may be a range having an upper limit or no upper limit, depending on the variable being defined). For example “at least 1” means 1 or more than 1, and “at least 80%” means 80% or more than 80%.
(6) The term “at most” followed by a number is used to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%.
(7) A range written as “(a first number) to (a second number)” or “(a first number)-(a second number)” means a range whose lower limit is the first number and whose upper limit is the second number. For example, “from 8 to 20 carbon atoms” or “8-20 carbon atoms” means a range whose lower limit is 8 carbon atoms, and whose upper limit is 20 carbon atoms.
(8) The terms “plural”, “multiple”, “plurality” and “multiplicity” are used herein to denote two or more than two features.
(9) When a method is described as comprising two or more defined steps, the defined steps can be carried out in any order or simultaneously (except where the context excludes that possibility), and the method can optionally include one or more other steps which are carried out before any of the defined steps, between two of the defined steps, or after all the defined steps (except where the context excludes that possibility).
(10) When reference is made to two or more features, this includes the possibility that the two or more features are replaced by a lesser number or greater number of features providing the same function (except where the context excludes that possibility).
(11) The numbers given should be construed with the latitude appropriate to their context and expression; for example, each number is subject to variation which depends on the accuracy with which it can be measured by methods conventionally used by those skilled in the art.
(12) Parts, ratios and percentages are by weight, except where otherwise noted.
(13) Temperatures are in degrees Centigrade (° C.).
(14) Molecular weights of polymers are in Daltons; are number average molecular weights (Mn) unless stated to be weight average molecular weights (Mw); and are measured by gel permeation chromatography (GPC) with a light scattering detection method, for example using a DAWN DSP laser photometer from Wyatt Technology, unless stated to be measured using GPC against a polystyrene standard.
(15) The terms “melting point” (often abbreviated to Tp), “onset of melting temperature” (often abbreviated to To) and “heat of fusion” (which is a measure of crystallinity of the polymer, is expressed in J/g and is often abbreviated to ΔH) are well known to polymer technologists and refer to quantities determined using a differential scanning calorimeter (hereinafter DSC), e.g. a Q 100 DSC from TA Instruments, at a rate of temperature change of 10° C./min, e.g. from −10 to 150° C. Tp is the peak melting temperature, To is the temperature at the intersection of the baseline of the DSC peak and the onset line, the onset line being defined as the tangent to the steepest part of the DSC curve below Tp, and ΔH is the heat of fusion associated with the endotherm or exotherm as calculated by the DSC and is reported in J/g. Unless otherwise stated, the values of Tp, To and ΔH are measured on the second heat cycle.
(16) Solubility parameters and polarity components thereof are calculated using the method described in D. W. van Krevelen, “Properties of Polymers” Elsevier, 1997, p. 200-214 especially p. 214 and reported in J^{1/2}/cm^{3/2},
(17) The term “associated” and grammatical variations thereof include any type of interaction, including chemical bonds (for example, covalent, ionic and hydrogen bonds) and/or Van der Waals forces, and/or polar and non-polar interactions through other physical constraints provided by molecular structure, and interactions through physical mixing.
(80) The term “alkyl” includes alkyl moieties which are straight chain alkyl moieties, branched chain alkyl moieties, cycloalkyl moieties, and moieties which consist essentially of two or more of straight chain alkyl, branched chain alkyl and cycloalkyl moieties.
(19) The term “bioerodable” (sometimes alternatively “biodegradable”) as applied to a CYC carrier or to a release composition means that the carrier or composition, when placed in or on the human body, is removed from the human body by the functioning of the body itself, the carrier being removed without change or as one or more lower molecular weight products resulting from the degradation of the carrier in the human body.
(26) Some of the structural formulas given below for CYC polymers show the repeating units in the general form

-(-unit#1-)ₓ-(-unit#2-)_{y}-

This representation is used to denote polymers in which the different repeating units are distributed randomly and/or are distributed in blocks containing only one of the repeating units. Thus, the polymers represented by these formulas can be either random copolymers or block copolymers.

The tables below sets out other abbreviations used in this specification, and the meanings to be attributed to them.

| Abbreviation | Meaning |
|---|---|
| AA | acrylic acid |
| MA | methacrylic acid |
| GA | glycolic acid |
| LA | lactic acid |
| PLGA | copolymer of GA and LA |
| PEG | polyethylene glycol |
| C18 | stearyl |
| C22 | behenyl |
| MCR | monomethacryloxypropyl alkyl polydimethylsiloxane |
| | wherein the alkyl group contains 1-8 carbon atoms. sold by |
| | Gelest as MCR-M17 |

The use of polymers in personal care applications, e.g. hair care, skin care, sun care, color care and body care has been disclosed for example in US patent and Patent Publications U.S. Pat. Nos. 6,540,984, 6,989,417, 7,101,832 and 7,175,832 (Landec Corporation); U.S. Pat. No. 5,736,125 (National Starch Corporation); U.S. Pat. Nos. 5,622,694, 5,662,892, 5,916,547, 5,919,439, 6,074,628 and 6,113,883 (Procter and Gamble Corporation); and U.S. Pat. Nos. 6,503,494, 6,572,869, 6,361,781, 6,569,409, 6,464,969, 6,565,839, 7,335,348, 6,789,550, 6,811,770, 6,949,504, 7,129,276, 7,255,870, 6,946,518, 7,090,420, 7,083,347, 2002041857, 2002127251, 2003039621, 2004005279, 2005188474, 2005191262, 2005172421, 2005191258, 2004180021, 2004191200, 2004228890, 2005031656, 2005013838, 2005142082, 2005123493, 2005008667, 2005031565, 2005169949, 2005169865, 2005261159, 2005175570, 2005180936, 2006078519, 2005287093, 2005287183, 2005287100, 2005287101, 2006078520, 2006130248, 2006233732, 2006216257, 2006292095, 2006263438, 2007134192, 2005137117 and 2003039671 (L'Oreal). The entire disclosure of each of those patents and patent publications is incorporated herein by reference for all purposes.

The polymers disclosed in those patents and publications include certain. SCC polymers which can be used to “structure” or thicken oils, both in anhydrous formulations (like lipsticks or underarm formulations), and in formulations containing an oil and a water phase, e.g. lotions, creams etc. those polymers, which are not bioerodable, function by associating in the oil phase to create a network. This results in thickened formulations having improving sensory properties (e.g. texture, touch and feel). The SCC polymers can also be used in hair care styling and restyling formulations, for example using an SCC polymer having a Tp such that the hair can if desired be repeatedly styled and restyled by heating/reheating.

The present invention provides additional materials which are useful ingredients of personal care compositions, and which can provide useful alternatives to the known materials. A particular advantage of some of the CYC carriers is that they are bioerodable, which makes them environmentally acceptable (“green”), particularly bearing in mind that personal care compositions are often disposed of through household sinks and drains. Often the bioerodable CYC polymers degrade to natural materials like lactic acid, glycolic acid, fatty acids, glycerol, etc. The components of bioerodable CYC polymers can be selected to exhibit longer or shorter degradation rates, e.g. by selection of the blocks in a block copolymer. When selecting a bioerodable CYC carrier for use in a cosmetic composition, e.g. to thicken an oil phase, a balance may need to be struck between the desirability of bioerodability and the ability of the bioerodable CYC carrier to provide desired functionality. In some cases, it may be desirable to combine a bioerodable CYC carrier with a non-bioerodable component to obtain the desired functionality, or to make use of a block copolymer which includes both biodegradable blocks and non-biodegradable blocks.

The CYC carriers suitable for use in personal care compositions include, but are not limited to, the bioerodable CYC polymers disclosed in detail herein, e.g. the following:—
(a) ECC-PLGA polymers—End capped crystalline PLGA copolymers containing 1, 2, 3, 4, 5, 6 or more Cy moieties.
(b) Alkyl polylactide polymers in which the methyl group of the lactide monomer has been substituted by Cy moieties.
(c) CYSC-PLGA block copolymers, in which the PLGA portion degrades leaving only a small amount of non-biodegradable polymeric units.
(d) Other polymers in which the main chain can be hydrolyzed (and is therefore bioerodable), for example

1) Polycarbonates
i. Side-chain crystalline dimethylol propionic acid (DMPA) polycarbonates
ii. Side-chain crystalline polycarbonates from glycerin carbonate
iii. Side-chain crystalline polycarbonates from monoglycerides, dimethyl carbonate, and optionally lactic/glycolic acid(s).

2) Polyesters
i. Side-chain crystalline malic, citric or tartaric acid polyesters
ii. Side-chain crystalline malic or tartaric acid PLGA polyesters
iii. Polyesters from glycidol, crystalline fatty acid and polybasic acid
iv. End-cap crystalline PLGA polyol polyesters
v. End-cap crystalline glycerin carbonate ECC-PLGA polyesters
vi. ECC-12-hydroxystearic acid PLGA polyesters
vii. Crystalline fatty ester dimethylol propionic acid polyesters

3) Side-chain Crystalline Oxazolines

4) Crystalline Alkyl Dioxanones

5) Side-chain Crystalline Hyaluronic acid

6) Side-chain Crystalline Cellulose

Personal care compositions can optionally also have one or more of the following features.

(a) The composition can contain a compound containing a long chain n-alkyl component, or other Cy moiety, which will “self assemble” with the biodegradable CYC polymer. Such compounds include for example fatty acids, fatty alcohols, modified PEG's, and compounds obtained by esterifying or otherwise modifying a polyhydroxy compound, e.g. sorbitol, by reaction with an n-alkyl carboxylic acid or acid derivative in which the n-alkyl group contains at least 14, preferably 18-30 or 18-22 carbon atoms.
(b) The composition can contain, in addition to the CYC biodegradable polymer, one or more other synthetic or naturally occurring biodegradable polymers, e.g. PLGA, poly(ethylene oxide), polypropylene oxide, poly(vinyl alcohol), polyurethane, collagen, gelatin, chitosan or sugar, and/or one or more other polymers which are not biodegradable, e.g. the SCC polymers already known for use as a oil thickeners.
(c) The composition can contain additives, which may be inorganic or organic, e.g. kaolin, talc, magnesium trisilicate, and various derivatives of cellulose.

Personal care compositions of the invention can be of any type, including those disclosed in the patents and patent applications incorporated by reference herein. They can be used, for example, in any personal care application on the face, body, hair, eyelashes, lips, skin, scalp, nails, etc. They can, for example be deodorants, antiperspirants, lipsticks, makeups, lotions, creams, oil-in-water emulsions, water-in-oil emulsions, hair care, color care, skin care, sun care, hair styling, hair restyling, shampoos, conditioners, nail varnishes, gels and oils, creams, hand creams, night renewal creams, body milks and lotions, light facial creams, protective day creams, liquid moisturizing emulsions, and products designed to remove makeup/personal care products. The compositions can also contain any of a wide range of additives including those disclosed in the patents and patent applications incorporated herein by reference. The additives can for example be UV absorbers, fragrances, biocides, antimicrobial agents germicides, antioxidants, preservatives, disinfectants, enzymes, nutrients, minerals, and release materials (including pharmaceuticals which are active physiologically or pharmacologically, either topically or systemically).

### CYC Carriers

### Crystallinity and Heat of Fusion of CYC Carriers

The crystallinity which is an essential part of the CYC carriers is provided by association of the Cy moieties with each other and/or with moieties in other materials (which moieties may also be Cy moieties). The Cy moieties comprise moieties which can overlap and interact with each other and/or other moieties to form crystalline aggregates or domains. Examples of such moieties include polymethylene moieties containing at least 13, preferably at least 15, particularly at least 17, and up to 50 or even more, methylene moieties, e.g. 15-23 or 17-21 methylene moieties. Many other examples of such moieties are disclosed below. The extent of the crystallinity depends upon the ability of such moieties to overlap and interact. Crystallinity is, therefore, increased by increasing the proportion of Cy moieties, by increasing the proximity of the Cy moieties to each other (for example by placing all or a large proportion of the Cy moieties in a block (e.g. a grafted block) of a copolymer, or in a terminal unit of a polymer or compound), and by minimizing other moieties which are bulky enough to interfere with the ability of the Cy moieties to overlap and interact. In the limited context of the polymers known as side chain crystalline (SCC) polymers, it is known to produce crystallinity by the interaction of polymethylene moieties and the like. Those skilled in the art will have no difficulty, having regard to the disclosure of this specification and their own knowledge, in making and using the crystalline CYC carriers which are useful in this invention.

Patents and other publications relating to SCC polymers include J. Poly. Sci. 60, 19 (1962); J. Poly. Sci, (Polymer Chemistry) 7, 3053 (1969), 9, 1835, 3349, 3351, 3367, 10, 1657, 3347, 18, 2197, 19, 1871; J. Poly. Sci, Poly-Physics Ed 18 2197 (1980); J. Poly. Sci, Macromol. Rev, 8, 117 (1974); Macromolecules 12, 94 (1979), 13, 12, 15, 18, 2141, 19, 611; JACS 75, 3326 (1953), 76; 6280; Polymer J 17, 991 (1985); and Poly. Sci USSR 21, 241 (1979); U.S. Pat. Nos. 4,830,855, 5,120,349, 5,129,180, 5,156,911, 5,254,354, 5,387,450, 5,412,035, 5,469,867, 5,665,822, 5,752,926, 5,783,302, 6,013,293, 6,060,540, 6,199,318, 6,210,724, 6,224,793, 6,255,367, 6,376,032, 6,492,462, 6,540,984, 6,548,132, 6,831,116, 6,989,417, and 7,101,928; and US Patent Application Publications Nos. 2001/0018484, 2002/0090425 and 2002/0127305. The entire disclosure of each of those publications, patents and patent publications is incorporated herein by reference for all purposes.

The value of the heat of fusion, ΔH, of a CYC carrier reflects the extent of its crystallinity, and is at least 3, for example at least 4, at least 10, at least 20, at least 25, at least 30, at least 35, at least 40, or at least 45, J/g., and may be for example 3-50, 4-50, 10-50, 10-40, 15-35, 3-30, 3-22, or 3-10, J/g. The CYC carrier can, but does not need to, and generally does not, have so-called “main chain crystallinity”, i.e. crystallinity resulting from crystallization of the polymer backbone. Thus, in some embodiments, the CYC carrier have main chain crystallinity, for example when a main chain crystalline polymer is modified to include b-Cy moieties at intermediate and/or terminal points. If the CYC carrier does have main chain crystallinity, Tp and ΔH should be assessed solely on the crystallinity provided by the Cy moieties, ignoring the Tp and ΔH resulting from the main chain crystallinity.

### Tp, Melting Range (Tp−To) and Mn of CYC Carriers.

The melting point (Tp) of the CYC carriers is primarily dependent on the nature of the Cy moieties, and (unlike main chain crystalline polymers) is not highly dependent on the molecular weight of the carrier. The CYC carrier can for example have a Tp of 0-105, 22-70, 35-50, 38-50, 37-42, or 40-47° C., or at least 37° C. Tp is often 22-70° C., but CYC carriers with a Tp below 22° C., for example as low as 2° C., or higher than 70° C., e.g. up to 105° C., may be useful in certain embodiments. The fact that the molecular weight can be controlled with relatively little change in Tp and Tp−To is important. It means that it is possible, by selection of the Cy moieties (and other moieties) in the CYC carrier, and the method used to prepare the CYC carrier (e.g. making use of chain transfer agents when preparing a polymer), to make a carrier which has a desired Tp, Tp−To and molecular weight. These measures can also be used to control the melting range of the CYC carriers. The melting range can conveniently be quantified by the value of Tp−To (To being the onset of melting temperature, as defined above). In some embodiments, the CYC carrier has a Tp−To<Tp^{0.7}, e.g. <Tp^{0.6}; for example, in this embodiment, if Tp is 40° C., Tp−To is less 13.2° C., e.g. less than 9.1° C. In other embodiments, Tp−To is less than 15° C., less than 10° C. or less than 5° C. A relatively low melting point, and a relatively small, melting range is advantageous, in that it makes it easy to remove the cosmetic composition from the skin, hair or other substrate, simply through the use of hot water.

The ability to control these variables is valuable for various purposes. For example, in the case of a cosmetic formulation, Tp can be selected with reference to in vivo temperatures, and the temperature at which the composition is to be removed. In addition, Tp and Tp−To can be selected to facilitate the preparation and processing of release compositions at relatively low temperatures, particularly when the release material would be degraded by higher temperatures.

Generally, the Tp of a CYC polymer increases as the number of linear carbon atoms in the Cy moieties increases. For example, the homopolymers of n-alkyl acrylates in which the n-alkyl group contains 14, 16, 18, 22, 30, 40 and 50 carbon atoms have Tps of about 20, 36, 49, 65, 76, 96 and 102° C. respectively; and the homopolymers of the corresponding n-alkyl methacrylates have Tps of about 10, 26, 39, 55, 68, 91 and 95° C. respectively. The Tp of a copolymer consisting of two or more -Y(b-Cy)- moieties reflects the relative proportions of the different moieties. Random copolymers of long chain n-alkyl acrylates and n-alkyl methacrylates generally have intermediate Tps in the range of 0 to 85° C. dependent on the length of the n-alkyl chain. The presence of other moieties (i.e. moieties which do not contain Rc moieties) generally reduces Tp and broadens the melting range. Random copolymers with other monomers, e.g. acrylic acid or butyl acrylate, typically have somewhat lower melting temperatures. Longer chain Rz moieties generally depress Tp more than shorter chain Rz moieties, because longer chain moieties have greater potential to disrupt the formation of crystalline domains by the Cy moieties.

The Mn of the CYC carrier can influence the incorporation and/or retention and/or delivery of a release material. Mn can for example be 500-1,000,000, e.g. 1,000-50,000, 2000-40,000, 2000-25,000, 2000-30,000, or 3000-20,000, or 3000-10,000, or 3000-8000. In some cases, it is less than 200,000, or less than 100,000, or less than 80,000, or less than 60,000, or less than 50,000, or less than 30,000, or less than 25,000, or less than 20,000, or less than 10,000, or less than 8000, or less than 7000, or less than 5000, or less than 2500, or less than 1000, e.g. 1,000-20,000, or 1,000-10,000 or 2,000-20,000, or 3,000-5,000. In other cases, it may be greater than 1,000,000. In yet other cases, e.g. when the CYC polymer is a cross-linked hydrogel, it may be infinite. In some embodiments, the CYC carrier has a molecular weight less than 20,000, or less than 15,000 or less than 10,000. In some embodiments, Mn is greater than 600, or greater than 800, or greater than 1000. In some embodiments, the CYC carrier is not charged or crosslinked, so that it can be voided from the body.

### Different Types of CYC Polymer

The CYC polymer can be a homopolymer or a copolymer. If it is a copolymer, it can be a random copolymer, a graft copolymer or a block copolymer (including a thermoplastic elastomer), or a core shell polymer. For example, the polymer can

(a) comprise one or more types of -Y(Rc) moiety and one or more types of -Z(Rz)- moiety, all the moieties being randomly distributed;
(b) be a block copolymer comprising (i) polymer blocks consisting essentially of one or more -Z(Rz)- moieties, and (ii) polymer blocks which comprise one or more types of repeating unit of the formula -Y(Rc)-, and optionally one or more types of repeating units of the formula -Z(Rz)-; or
(c) be a graft polymer, for example (i) a polymer comprising a backbone which comprises, or consists essentially of, one or more -Y(Rc)- moieties, and grafted side chains each of which comprises, or consists essentially of, one or more -Z(Rz)- moieties, or (ii) a polymer comprising a backbone which comprises, or consists essentially of, one or more -Z(Rz)- moieties, and grafted side chains each of which comprises, or consists essentially of, one or more -Y(Rc)- moieties.

In some embodiments, it is preferred to use a CYC carrier which is bioerodable (as defined above).

It is generally preferable that the CYC carrier be substantially physiologically inactive.

### Y and Z Moieties

The backbone of a CYC polymer can be of any kind. The -Y- moieties (which will be present in a CYSC polymer and will optionally be present in an ECC polymer) and the -Z- moieties (which will be present in an ECC polymer and will optionally be present in a CYSC polymer) can be the same as, or different from, each other. The -Y- moieties and/or the -Z- moieties can for example comprise carbon atoms which are linked to each other directly by covalent bonds or through other elements or combinations of elements, and repeating units can be linked to each other directly by covalent bonds or can contain linking units comprising one or more atoms, e.g ester (including orthoester), amide, ether or phosphate linkages. For example, the CYC polymer can consist essentially of, or can comprise, sections which consist essentially of, polyacrylates, poly-alkyl acrylates, poly-fluoroacrylates, polymethacrylates, polyalkyl methacrylates, poly-N-alkyl methacrylamides, poly-alkyl oxazolines, poly-alkyl vinyl ethers, poly-alkyl 1,2-epoxides, poly-alkyl glycidyl ethers, poly-vinyl esters, poly-acrylamides, poly-methacrylamides, poly-maleimides, poly-α-olefins, poly-p-alkyl styrenes, poly-alkyl vinyl ethers, polyolefins, polyethers, polyurethanes, polysilanes, polysiloxanes, or poly(alkyl phosphazenes). CYSC polymers can for example be obtained directly by addition polymerization of suitable monomers, e.g. acrylic, methacrylic, olefinic, epoxy, esters, amides, vinyl or silicon-containing monomers. ECC polymers can for example be obtained by modification of the terminal units of polyesters obtained for example by the polymerization of monomers such as glycolic acid and lactic acid. Further details of CYC polymers and methods for preparing CYC polymers are given below.

In some embodiments, the backbone of a CYC polymer includes a plurality of bioerodable linkages. In such CYC polymers, a plurality of the -Y- moieties and/or the -Z- moieties can for example have the formula

-D_{nd}-Q-Eₙₑ- (Q1)

where
Q is —O—, —NH— or —S—,
nd is 0 or 1,
ne is 0 or 1,
D is —CO—, and
E is —CO— or —CO—O—
   For example, in an ether linkage, nd is 0, ne is 0 and Q is —O—; in an ester linkage, nd is 1, ne is 0, D is —CO— and Q is —O—; in an anhydride linkage, nd is 1, ne is 1, D is —CO—, E is —CO—, and Q is —O—; and in a carbonate linkage, nd is 0, ne is 1, E=—CO—O—, and Q is —O—. Other biodegradable linkages are those present in polyorthoesters, which have the structure:

—C(OR¹)(OR²)(OR³)—

where two of R¹, R² and R³ are part of the polymer backbone.

### b Moieties

b is a bond or a moiety linking the Cy moiety to an intermediate point on the polymer backbone or to a terminal moiety. Thus, b may for example be a covalent bond, or a divalent organic moiety (e.g. an aliphatic, aromatic or mixed aliphatic/aromatic moiety) or inorganic moiety. Examples of b moieties include ester (i.e. —COO—), carbonyl, amide, amine oxide, hydrocarbon (for example phenylene), amino, ether, polyoxyalkylene, and ionic salt linkages (for example a carboxyalkyl ammonium, sulfonium or phosphonium ion pair).

### Cy Moieties

The Cy moieties (which provide chains pendant from an intermediate location and/or from a terminal location of a CYC carrier) in a particular CYC carrier may be the same or different. The Cy moieties must be such that they are capable of interacting with other Cy moieties, for example other Cy moieties elsewhere on the same carrier and/or on a different compound, which may be a polymer (which may or may not be a CYC polymer) and/or on a non-polymeric compound, to provide crystallinity. The interaction between the Cy moieties is generally through hydrogen bonds or Van der Waals forces, rather via covalent or ionic bonding.

The Cy moieties can be of any kind, for example aliphatic, e.g. alkyl, or mixed aliphatic aromatic. The CYC carriers contain Cy moieties such that the carrier, when examined on a DSC in the manner defined below, has a heat of fusion of at least 4 J/g and a Tp of at least 0° C. resulting from crystallization of the CYC moieties. Some CYSC polymers having these characteristics are known and have been referred to by those skilled in the art as side chain crystalline polymers (sometimes abbreviated to SCC polymers or SCCPs).

The Cy moieties often comprise a linear carbon chain of at least 8 or at least 12 carbon atoms directly linked to each other, e.g. 12-50 or 16-30 carbon atoms. The moiety is generally not branched, but can be branched providing that the branching does not prevent the moiety from being capable of crystallization. Similarly, the moiety can be unsubstituted or substituted predominantly only by fluorine atoms, or can be substituted by other moieties which do not prevent the moiety from being capable of crystallization.

Cy can be for example a moiety comprising 6 to 50, e.g. 12 to 50, preferably 12 to 22 or 16 to 22, substantially linear carbon atoms, e.g. a moiety comprising at least 11 methylene moieties, for example 11-49 methylene moieties and a terminal methyl moiety, or a moiety comprising at least 5, e.g. 5 to 49 linear perfluoro or substantially perfluoro methylene moieties and a terminal perfluoromethyl moiety or hydrogen atom. Specific examples of suitable Cy moieties include C14, C16, C18, C20, C22, C30, C40 and C50, in particular n-alkyl moieties containing 14, 16, 18, 20, 22, 30, 40 and 50 carbon atoms, and partially or fully fluorinated n-alkyl groups containing at least 8 carbon atoms, and mixtures of Cy moieties having similar average chain lengths.

### Cy Moieties Containing Polyoxyalkylene Moieties

Some useful Cy moieties include polyoxyalkylene, e.g. polyoxyethylene, units. Such a Cy moiety can for example be derived from alkoxy polyoxyalkylene(meth)acrylates, where the alkyl portion of the alkoxy group is preferably an alkyl, particularly an n-alkyl, group containing 12 to 50, preferably 12 to 22 carbons, and the polyoxyalkylene unit is a homopolymer, random copolymer, or block copolymer containing 2 to 100, e.g. 5 to 100, preferably 5 to 60, oxyalkylene units, preferably 2-20, e.g. 2-4, oxyalkylene units. Specific examples of such monomers include cetyl polyethoxylated methacrylate, stearyl polyethoxylated (meth)acrylate, behenyl polyethoxylated (meth)acrylate, lauryl polyethoxylated (meth)acrylate, cholesterol polyethoxylated (meth)acrylate and the like. The polyoxyalkylene unit can be attached to the alkyl side chain portion, as for example in hydroxypolyalkyleneoxyalkyl (meth)acrylates with similar alkyl and polyalkyleneoxy groups as above, e.g. hydroxypolyethleneoxystearyl acrylate, hydroxypolyethyleneoxycetyl methacrylate and the like.

All the moieties of the formula -Y(b-Cy)- in a CYC polymer can be the same, or there can be a plurality of (i.e. two or more) different types of moiety which differ from each other in one or more of Y, b and Cy. In some CYC polymers containing a plurality of different types of -Y(b-Cy)- moiety, the different types are randomly distributed throughout the polymer; in others, the different types are distributed in a desired non-random fashion in at least part of the polymer, such as in a block copolymer or a graft copolymer. For example, the polymer can comprise at least one polymer block which comprises only one type of repeating unit of a first formula -Y(b-Cy)- and a second polymer block which comprises only repeating units of a second formula -Y(b-Cy)-. Alternatively the polymer may comprise one or more sections which contain a plurality of different -Y(b-Cy)- moieties distributed randomly, and at least one polymer block which comprises (i) only repeating units of one or other of the different -Y(b-Cy)- moieties and/or a third -Y(b-Cy)- moiety, or (ii) a plurality of randomly distributed different repeating units of two or more of the first, second and third moieties.

When there are two or more different -Y(b-Cy)- moieties, the Cy moieties may have, for example, an average length of 6 to 50 linear carbon atoms, the average being calculated by adding all lengths of all the Cy moieties in the polymer (or, in the case of a block, including graft, copolymer, all the Cy moieties in the block) and dividing by the number of Cy moieties. The average length may have, for example, an accuracy of +/−3-10%, e.g. +/−5%.

A CYSC polymer can consist essentially of -Y(b-Cy)- moieties. However, many useful CYSC polymers contain less than 75%, or less than 50%, e.g. 1 to 75%, 5 to 50%, 15-50%, 15-30% or 10 to 25%, of -Y(b-Cy)- moieties, for example less than 1%, 3%, 5%, 7%, 10%, 15%, 20%, 25%, 30%, 40% or 50% of -Y(b-Cy)- moieties. Particularly at the lower end of the -Y(b-Cy)- moiety content, in order to enhance crystallinity, the Cy moiety preferably contains at least 18 linear carbon atoms and/or the -Y(b-Cy)- moieties are present as grafted chains or blocks which consist essentially of the -Y(b-Cy)- moieties.

As briefly noted above, CYC polymers often include other units, in addition to the repeating units of formula (1) and/or the terminal units of formula (2). As noted above, those other units can for example be represented by the formula

-Z(Rz)- (3)

where Z is a moiety forming part of the polymer backbone and Rz represents a monovalent moiety which does not comprise a Cy moiety. All the repeating units of the formula -Z(Rz)- can be the same, or there can be a plurality of different types of repeating unit which differ from each other in Z, or in Rz, or in both Z and Rz. The moieties of the formula -Z(Rz)- can be randomly distributed throughout the polymer, or they can be distributed in a desired non-random fashion in at least part of the polymer. The Z(Rz) moieties contribute to the chemical and other characteristics of the CYC polymer, and their presence can be valuable for this purpose. For example, many useful CYC polymers have an amphiphilic character, with the CYC moieties providing hydrophobic characteristics and the Z(Rz) moieties providing hydrophilic characteristics. A detailed disclosure of the repeating units of formula (3) follows later in this specification.

Other examples of CYSC polymers consist essentially of or comprise atactic, syndiotactic and isotactic polymers of long chain n-alkyl α-olefins (e.g. the atactic and isotactic polymers of C16 olefin, having Tp's of 30° and 60° C. respectively); polymers of n-alkylglycidyl ethers (e.g. the polymer of C18 alkyl glycidylether); polymers of long chain n-alkyl vinyl ethers (e.g. the polymer of C18 alkylvinylether having a Tp of 55° C.); polymers of long chain n-alkyl-α-epoxides (e.g. the polymer of the C18 alkyl α-epoxide having a Tp of 60° C.); polymers of long chain n-alkyloxazolines (e.g. the polymer of C16 alkyl oxazoline having a Tp of 155° C.); polymers obtained by reacting an hydroxyalkyl acrylate or methacrylate with a long chain alkyl isocyanate (e.g. the polymers obtained by reacting hydroxyethyl acrylate with C18 or C22 alkyl isocyanate and having Tp's of 78° and 85° respectively); and polymers obtained by reacting a difunctional isocyanate, a hydroxyalkyl acrylate or methacrylate, and a long chain fatty alcohol (e.g. the polymers obtained by reacting hexamethylene diisocyanate, 2-hydroxyethyl acrylate, and C18 or C22 alcohols).

### -Z(Rz)- Moieties

In CYSC polymers, the -Z(Rz)- units are often derived from monomers that can be easily copolymerized with the monomers which provide the Cy-containing moieties of formula (1), with Rz being chosen to provide the CYSC polymer with desired properties, for example hydrophilic properties. For example, Z and Y_{ch} can both be derived from a monomer containing an ethylenic double bond, e.g. an ester or other derivative of acrylic or methacrylic acid.

An ECC polymer has a backbone which is made up of -Z(Rz)- units, and optionally units of formula (1). As described in detail below, in many ECC polymers, the Z moieties comprised bioerodable linkages, for example ester linkages. In ECC polymers, the Rz moieties (in contrast to their role in CYSC polymers) may simply be hydrogen atoms or other moieties which are not chosen to contribute to the physical or chemical properties of the polymer (though they may of course do so).

In both CYSC and ECC polymers, the repeating units of the formula -Z(Rz)- can be the same, or there can be a plurality of different types of repeating unit which differ from each other in Z, or in Rz, or in both Z and Rz. If the CYC polymer contains different repeating units (of either formula (1) or formula (2) or both), the repeating units can be randomly distributed throughout the polymer, or they can be distributed in a desired non-random fashion in at least part of the polymer.

### -Z(Rz)- Units in CYSC Polymers.

The presence of Z(Rz) moieties in a CYSC polymer generally depresses the melting temperature and reduces the crystallinity of the CYSC polymer, to an extent which is dependent on the proportion and distribution of the Z(Rz) moieties and the nature of the Z(Rz) moieties. The Z(Rz) moieties also contribute to the chemical and other characteristics of the CYSC polymer, and their presence can be valuable for this purpose. For example, many useful CYSC polymers have an amphiphilic character, with the Cy-containing moieties providing hydrophobic characteristics and the Rz moieties providing hydrophilic characteristics.

The Z(Rz) moieties in a CYSC polymers can be of any kind, for example aliphatic, e.g. alkyl, or mixed aliphatic aromatic. The Z(Rz) moieties can contain any suitable linking group through which they are linked to each other and to the Y(Rc) moieties. For example the polymer can comprise sections which comprise the Z(Rz) moieties and which are polyacrylate, polymethacrylate, polyalkyl (meth)acrylate, poly-N-alkyl acrylamide, poly-alkyl oxazoline, poly-alkyl vinyl ether, poly-alkyl 1,2-epoxide, poly-alkyl glycidyl ether, poly-vinyl ester, poly-acrylamide, poly-methacrylamide, poly-maleimide, poly-α-olefin, poly-p-alkyl styrene, poly-alkyl vinyl ether, polyolefin, polyether, polyurethane, polysilane, polysiloxane, or poly(alkyl phosphazene).

All the Z(Rz) moieties can be the same, or there can be two or more different Z(Rz) moieties, randomly distributed and/or arranged in a desired distribution, as for example in a block copolymer in which one of the blocks comprises essentially only one type of Z(Rz) moiety, and another of the blocks comprises essentially only another type of Z(Rz) moiety. The Z moieties (which, when there are two or more different types of Z moiety, can be the same or different) can for example be derived from the addition and/or condensation polymerization of suitable monomers, e.g. acrylic, methacrylic, olefinic, epoxy or vinyl monomers.

The bond between Z and Rz can be any bond as described for the bonds between Y and Rc. The bond may be hydrolytically stable, unstable, or labile to hydrolysis or enzymatic cleavage.

Suitable monomers from which Z(Rz) moieties can be derived can contain the desired Rz moieties, and/or can contain Rz precursor moieties some or all of which are converted into Rz moieties during or after the polymerization. Suitable monomers are for example alkyl (e.g. 2-ethylhexyl, butyl, ethyl, methyl) (meth)acrylates, hydroxyalkyl (meth)acrylates (e.g. hydroxyethyl acrylate, hydroxyethyl methacrylate) alkoxyalkyl (meth)acrylates (e.g. methoxyethyl acrylate, ethoxyethyl methacrylate), and hydroxypolyoxyalkylene (meth)acrylates (e.g.—hydroxypolyoxyethylene methacrylate or acrylate where the ethyleneoxy units are from 4 to 50), other (meth)acrylates (e.g. glycidal methacrylate, (acetoacetoxy)ethyl methacrylate), acrylamides and methacrylamides; styrene; monoacrylic functional polystyrene; alkyl vinyl ethers, and alkyl vinyl esters; and in all of which monomers the alkyl groups are alkyl groups which are not Rc moieties, for example n-alkyl moieties containing less than 12, e.g. 1, 2, 3, 4, 5, 6, 7, 8, 9, 10, 11, or 12 carbon atoms (e.g. vinyl laurate); and polar monomers, for example acrylic acid, methacrylic acid, itaconic acid, dimethylaminoethyl methacrylate, diethylaminoethyl methacrylate, t-butyl acrylamide, dimethylaminopropyl methacrylamide, N-isopropyl acrylamide, acrylonitrile, methacrylonitrile, maleic anhydride, monobutyl fumarate, vinyl acetate, N-vinyl pyrrolidone, and comonomers containing amine groups.

In certain embodiments Rz may comprise polyoxyalkylene e.g. polyoxyethylene, moieties, for example a polyoxyalkylene moiety which links the Z moiety to an end group which is not an Rc moiety

The Rz moieties in CYSC polymers can for example include one or more desired functional groups, including, but not limited to, the functional groups forming part of the compounds listed below (the disclosure of those functional groups being independent of the moiety forming the remainder of the listed compound).

In some embodiments, the CYSC polymer includes Z(Rz) moieties that can form a covalent bond with the release material. In such embodiments, the extent of covalent linkage between the polymer and the release material will be an important factor in determining release characteristics of the release composition.

In some embodiments, the CYSC polymer comprises functional groups to assist in the transport of the release composition into a biological system, or to assist in the adherence of the composition to deliver the release material. For example the composition can include mucoadhesives sites, dermal adhesives, or large molecules that enhance bioavailability and reduce immunogenicity, for example, polyoxyalkylene vinyl monomers, e.g., with PEG or PEGylated groups. These PEGylated groupings in the CYC polymer will typically be introduced via comonomers,

In some embodiments, the CYSC polymer includes units comprising ionic groups, e.g. units derived from ionic vinyl monomers. The ionic groups can help to stabilize the polymer formulation, Other embodiments may employ for example, PEGylated monomers or acidic or other non-ionic or monomers respectively that may be incorporated together as part of the same CYSC polymer or may be present in separate CYSC polymers to be mixed with a release material.

In some CYSC polymers, the Z(Rz) moieties may also enhance the physical surface properties of the release composition. For example, polyoxyalkylene units, e.g. polyoxyethylene (meth)acrylate units, can provide beneficial slip or hydrophilic properties, for example to stabilize embodiments forming part of a cosmetic composition. In some embodiments, the Z(Rz) moieties may also help in the sustained release or delivery of the release material.

CYC carriers that contain cationic groups can be obtained through the use of monomers which contain cationic groups or groups that can be subsequently modified to provide cationic groups. For example, an acid-ECC PLGA can be reacted with a dialkyl alkanol amine or dialkypolyoxyalkylene amine, and the resulting product converted to a cationic product through quaternization or to an amine oxide product through peroxidation. Quaternary cationic CYC carriers can provide useful skin bonding or hell bonding causes which promote a desirable touch and/or sheen or other desirable cosmetic qualities.

CYC carriers comprising multivalent ions, for example, calcium, zinc or magnesium, can be produced by modification of CYC carriers containing carboxylic acid groups, thus producing an ion-cross-linked network. Such a network can be useful to entrap and carry a cosmetic material for hair care or skin care.

CYC carriers including silicon containing groups, e.g. siloxane groups, can be produced by the use of suitable comonomers or by modification of base polymers, e.g. CYSC, polyacrylates, and Cy ECC PLGAs.

Useful Rz moieties in CYSC polymers include:

(1) Nitrogen-containing side chains, for example the moieties which result from the polymerization of the groups of monomers and specific monomers listed below. It is noted that the identified Rz moieties and/or the functional groups thereon can also be obtained through the use of other monomers: N,N-dialkyl amino (in particular, dimethylamino) (meth)acrylates; ammonium salt-containing (meth)acrylates, for example 2-trimethylammonium methylmethacrylate chloride, methacrylamidopropyl trimethylammonium chloride, N,N-(diethyl or dimethyl)aminoethyl(meth)acrylate methosulfate; N-vinylpyrrolidinone;
imides like the ring-closed reaction products of maleic or itaconic anhydride with primary amines; 2-methacryloxy-N-ethylmorpholine; n- or t-butylacrylamide; (meth)acrylamide;
dimethylaminopropyl methacrylamide; 2-t-butylaminoethyl methacrylate; (meth)acrylonitrile; t-butylaminoethyl (meth)acrylate; acryloylmorpholine; N-(2-hydroxyethyl)acetamide; 1-piperidinoethyl (meth)acrylate; and amine oxide containing monomers obtained by reacting alkyl amine containing side chain containing monomers with an oxidizing agent to give an amine oxide of the precursor alkyl amine.

In certain specific embodiments, the formulations of the invention specifically exclude Rz side chains derived from N-vinylpyrrolidinone.

(2) Oxygen-containing side chains, for example the moieties which result from the polymerization of the groups of monomers and specific monomers listed below, including carboxyl- and sulfonic acid-containing monomers and salts thereof. It is noted that the identified Rz moieties and/or the functional groups thereon can also be obtained through the use of other monomers: acrylic acid, methacrylic acid; itaconic anhydride; itaconic acid; maleic anhydride; maleic acid; fumaric acid; monoesters and monoamides of fumaric acid, maleic acid, crotonic acid, and 2-acrylamido-2-methylpropane sulfonic acid (“AMPs”); vinyl sulfonic acid; hydroxyalkyl (meth)acrylates, in particular, hydroxyethyl, hydroxypropyl, and hydroxybutyl) (meth)acrylates; tetrahydrofu rfu ryl (meth)acrylate; glycidyl methacrylate; alkoxyalkyl (meth)acrylates, e.g. methoxyethyl (meth)acrylate; hydroxycaprolactone acrylate; 1-acryloxy-2-hydroxy-3-phenoxypropane; methylol methacrylate; ethoxyethyl (meth)acrylate; 2-(2-ethoxyethoxy)ethylacrylate; acetoacetoxyethyl (meth)acrylate; phenoxyethyl (meth)acrylate; (meth)acrolein; alkoxy or hydroxyl (polyoxyalkylene) alkyl (meth)acrylates, e.g. methoxy- or hydroxypolyoxyethylene (meth)acrylates, for example those in which the moles of ethyleneoxy units are from 2 to 80, preferably 6 to 50; alkoxy- or hydroxypolyoxypropylene-polyoxyethylene alkyl (meth)acrylates, for example those in which the blocks of each oxyethylene and oxypropylene unit are present in 1/1 to 1/3 ratios whereby the amount of oxyalkylene units in each block is 5 to 100, preferably, 5 to 60 units.
(3) Fluorine-containing side chains, for example the moieties which result from the polymerization of the groups of monomers and specific monomers listed below. It is noted that the identified Rz moieties and/or the functional groups thereon can also be obtained through the use of other monomers: trifluoroethyl (meth)acrylate; heptadecafluorodecyl (meth)acrylate; octafluoropentyl (meth)acrylate; eicosafluoroundecyl (meth)acrylate; hexadecafluorononyl (meth)acrylate; and tetrahydroperfluorodecyl (meth)acrylate.
(4) Phosphorus-containing side chains, for example the moieties which result from the polymerization of the monomers listed below and similar monomers. It is noted that the identified Rz moieties and/or the functional groups thereon can also be obtained through the use of other monomers: 2-methacryloyloxyethyl phosphoryl choline; 2-acryloyloxypropyl phosphoryl choline; and stearyl fumaroylethyl phosphoryl choline.
(5) Silicon-containing side chains for example the moieties which result from the polymerization of the groups of monomers and specific monomers listed below. It is noted that the identified Rz moieties and/or the functional groups thereon can also be obtained through the use of other monomers: silyl monomers, e.g. trimethylsiloxy ethyl(meth)acrylate, 3-acryloxypropyl trimethoxysilane, and 3-acryloxypropyl tris(trimethylsiloxy)silane, monomethacryloxymonotrimethylsiloxyterminated polyethylene oxide, monomethacryloxypropyl alkyl polydimethylsiloxane where the alkyl group contains 1-8 carbon atoms, preferably 1 or 4 carbon atoms, and similar materials sold by for example Gelest as MCR-M17 and MCR-M11, and the like.
(6) Ligand groups which bind to target receptor sites. Ligands can be physically mixed as well as being part of the CYC polymer.

Some CYSC polymers include Z(Rz) moieties in which at least some of the Rz moieties are hydrophilic, the CYC polymer then being an amphiphilic copolymer having both hydrophobic and hydrophilic characteristics. Formulations comprising such amphiphilic polymers may form micelles or emulsions or liposomes in water, for example containing a hydrophobic release material within the hydrophobic core. It is often convenient to provide CYSC polymers with hydrophilic character by the inclusion of polyoxyethylene oxide units (“pegylation”).

Some CYSC polymers include Z(Rz) moieties in which all the Rz moieties are hydrophobic, in which case the CYSC polymer will be a copolymer having only hydrophobic characteristics.

The CYSC polymer generally contains Z(Rz) moieties in amount less than 95%, generally less than 70%, for example less than 50%, e.g. 5 to 25%, based on the weight of the polymer (e.g. about 5, 7, 10, 15, 17, 20, 23 or 25%).

### Cross-Linked CYSC Polymers and CYSC Polymer Gels.

The CYC polymers are generally not cross-linked. However, in some cases, cross-linking is useful, and can be achieved in many ways. For example the CYSC acrylates may be crosslinked by small amounts of multifunctional monomers to give hydrogel like products which provide thickening and gelling properties to skin care formulations and hair products. Likewise, the CYC polymers, for example PLGA, can be crosslinked by multifunctional crosslinking agents, to give products which are useful, for example in stabilizing suspensions of pigments or other solids such as nanoparticle solids, e.g. those used in sunscreen formulas, for example, nanocrystalline zinc oxide or titanium oxide. A graft copolymer prepared by grafting a Cy PLGA product onto a CYSC acrylate, or vice versa, may provide a thickening and/or gelling to a skin care or hair care formulations.

Alternatively a cross-linked CYC polymer can be prepared by combining the non-cross-linked polymer with a release material, and then adding a cross-linking agent to set the mixture in a particular shape.

Gels and hydrogels can comprise a cross-linked CYSC polymer. Also, in some embodiments, the composition comprises a crosslinked CYSC polymer which, because a cross-linked polymer at least partially retains its shape even under conditions which would cause otherwise melting or swelling, e.g. above Tp, tends to hold a release compound longer than the corresponding non-crosslinked polymer. Some embodiments specifically exclude CYSC polymers which are cross linked or immobilized on a support so that they cannot flow at temperatures above their melting temperature.

In some embodiments, a CYSC polymer has a gel structure.

A gel structure can be provided by including a cross-linking multifunctional monomer in the monomer mixture used to prepare the CYSC polymer. Such monomers are best employed in emulsion polymerizations, since, in other types of polymerization, they can result in viscosities which are difficult to handle. Exemplary crosslinking monomers are ethylene glycol dimethacrylate, butylene glycol dimethacrylate, trimethylol propane triacrylate, hexane diol diacrylate and the like. These crosslinking monomers are generally employed only in small amounts, e.g. less than 0.5%, or less than 1%, such as 0.1% to 5%, or 0.2 to 2%.

Hydrogels can be hydrophobic, or amphiphilic, and they can be ionic or non-ionic. Non-ionic hydrogels swell when they absorb water. Ionic hydrogels, which can be anionic or cationic, can be caused to swell to varying degrees by a change in pH. An alkaline pH causes swelling of an anionic gel (because ionic groups like carboxyl are ionized at high pH), whereas a low pH causes swelling of cationic gels. These facts, optionally combined with the response of CYSC carriers to changes in temperature around Tp, can be utilized to control release of a release material, and provide a powerful tool for making compositions which will release a release material at a desired location.

In another embodiment, a solid powdered hydrogel is mixed with a solid powdered release material, and then hydrated for administration.

In another embodiment, a CYSC polymer containing (in addition to the -Y(b-Cy)- units) units derived from a N-iso-propyl acrylamide comonomer, either alone or in combination with units derived from acrylic and/or methacrylic acid, can form an amphiphilic hydrogel which can associate with (depending on the percentage of the hydrophobic and hydrophilic portions) either a hydrophobic or a hydrophilic release material.

In another embodiment, the CYSC polymer is in the form of a non-ionic hydrogel prepared by polymerizing a monomer component comprising a Cy-containing monomer and a neutral hydrophilic comonomer, for example, hydroxyethyl methacrylate, together with a small portion of a crosslinking agent, e.g. ethylene glycol dimethacrylate. In a modification of this embodiment, polyoxyethlene glycol methacrylate monomer is also included, thus adding to the gel structure and providing possible antithrombogenic properties to a pharmaceutical formulation. This polymer is also an amphiphilic polymer. As such it can be compatible with release materials which are either or both hydrophilic and hydrophobic.

In another embodiment, the CYSC polymer is in the form of a neutral hydrogel prepared by polymerizing a Cy-containing monomer, acrylamide and t-butyl acrylamide, together with a small amount of methylene bis acrylamide. Such a hydrogel can associate with a hydrophilic or hydrophobic release material.

In another embodiment, the CYSC polymer is in the form of a hydrogel prepared by polymerizing a Cy-containing monomer, acrylic acid and a block polyoxypropylene/polyoxyethylene/polyoxypropylene ester of acrylic or methacrylic acid. The resulting amphiphilic polymer, dependent on the block structure of the oxyalkylene ester, will absorb varying amounts of either or both of a hydrophilic and a hydrophobic release material. These release materials may be released slowly dependent on the hydrophobe/hydrophile balance of the oxyalkylene ester and the hydrophobe from the SCC monomer.

In another embodiment, a CYSC polymer and a release material are formulated as a solution which is of low viscosity before administration, but which, when exposed to another pH, forms a hydrogel which provides an in-situ gel reservoir for release of the release material dependent upon the hydrophobic and hydrophilic properties of the CYSC polymer.

### CYC Polymers in the Form of Emulsions

In some embodiments, the CYC polymer is in the form of an emulsion. The average size of the particles in the emulsion (and preferably the maximum size of substantially all particles) is preferably less than 1200 nm, e.g. less than 800 nm or less than 500 nm, for example less than 200 nm or less than 100 nm (0.1μ). In many embodiments, the size of emulsion particles is 50-200 nm, or 50-500 nm, or 100-1000 nm. Some such emulsions can be prepared using the techniques described in U.S. Pat. Nos. 6,199,318 and 6,540,984, the entire disclosures of which are incorporated herein by reference for all purposes. In general, injectable emulsion particles have a diameter of less than 800 nm, or they can be less than 20μ, or less than 10μ, or less than 1μ in size.

### Mixtures of CYC Carriers

A single CYC carrier or a mixture of CYC carriers can be used. A mixture of CYC carriers can provide a CYC assembly. The CYC carrier or carriers can also be mixed with an additional material, for example a polymer which is not a CYC polymer. The additional material can form a CYC assembly. Examples of such additional materials include main chain crystalline polymers, and bioerodable polyesters. Specific examples of such other polymers, particularly for use in combination with the ECC polymers, include poly(epsilon-caprolactone (PCL), poly(dioxanone), polyhydroxybutyrate (PHB), polyhydroxyvalerate (PHV), polyhydroxyalkanoates (PHAs), polyesters from 3-hydroxypropionic acid, polymers derived from methylene carbonate, polyanhydrides, polyorthoesters, naturally occurring polymers or their hydrolysis or degraded products such as sugars, hydrolyzed starches, hyaluronan (also called hyaluronic acid or hyaluronate), chitan, chitosan, and alkyl polylactides, including those disclosed in WO 2007/0142461 (Baker et al.), U.S. Pat. No. 6,469,133, US published application Nos. 20070142461 and 20010044514, the disclosures of which are incorporated herein by reference for all purposes.

The criteria for the selection of a particular CYC polymer or mixture of CYC polymers, and optionally one or more additional polymers, depend upon the release material and its desired loading and/or release, as further discussed below. Some embodiments of the invention make use of a composition containing a mixture of two or more CYC polymers having substantially different Tps, for example Tps which differ from each other by at least 2° C., or at least 4° C. or at least 6° C. or at least 8° C. or at least 10° C. For example, the composition may contain one or more CYC polymers melting at about 37° C. and one or more other CYC polymers melting at about 39° C. and/or one or more other polymers melting at about 41° C. Other embodiments make use of mixtures of CYC polymers having a release material bound through a range of ionic strengths as defined by the pKa or pKb of the release material-polymer pair.

### CYSC Acrylate Polymers

Some embodiments of the invention make use of CYSC polymers which are polyacrylates, for example poly(meth)acrylates. In poly(meth)acrylates, the Y_{ch} moieties, and generally the Z moieties, if present, have the formula

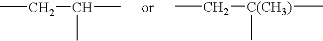

Such polymers preferably comprise units derived from n-alkyl (meth)acrylates in which the n-alkyl group contains at least 16, e.g. 16-50, e.g. 18-22 carbon atoms.

For example, in one embodiment, the CYSC polymer comprises one or more polymers selected from the group consisting of (1) polymers which consist essentially of units derived from at least one n-alkyl acrylate or n-alkyl methacrylate wherein the n-alkyl group contains 16-30 carbon atoms, and (2) polymers which consist essentially of units derived from at least one n-alkyl acrylate or n-alkyl methacrylate wherein the n-alkyl group contains 16-30 carbon atoms, and units derived from at least one acrylate or methacrylate comonomer containing one or more functional moieties selected from the group consisting of carboxyl, hydroxyl, alkoxypolyalkylene, hydroxypolyalkylene and amino moieties.

Other acrylate polymers contain units derived from (meth)acrylate monomers obtained by modifying very long chain mixtures of aliphatic alcohols, e.g. the Unilin alcohols sold by Baker Petrolite, in which the n-alkyl radicals average C30 or C40 or C50 carbon atoms. Such acrylate polymers can have Tps about 80, 90 or 100° C. respectively. Other acrylate polymers include polymers of long chain n-alkyl oxycarbonylamido-ethylmethacrylates (e.g. the polymers of C18 IEMA, C22 IEMA and C30 IEMA, having Tp's of 56° C., 75° C. and 79° C. respectively); and polymers of medium and long chain n-fluoro alkyl acrylates (e.g. the polymer of C8 hexadecafluoroalkylacrylate and the polymer of a mixture of C8-12 alkyl fluoroacrylates, having Tp's of 74° C. and 88° C. respectively).

### Other CYSC Polymers

Other CYSC Polymers include polycarbonates, polyesters, polyester oxazolines and polydioxanones, as described for example below

### CYSC Polycarbonates

Side-chain crystalline dimethylol propionic acid polycarbonates:

As an item of commerce used in water dispersible polyurethanes, dimethylol propionic acid (DMPA) is used as a polyol acid. When this acid is first reacted at the carboxylic acid position with a crystalline fatty alcohol, stearyl alcohol, for example, the crystalline diol ester can then be reacted with dimethyl carbonate to give the crystalline trimethylene carbonate analog which is reacted with trimethylene carbonate at different molar ratios to give a side chain crystalline polycarbonate:

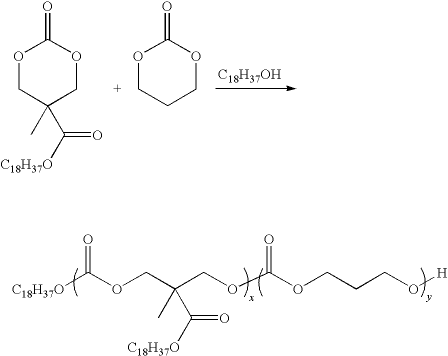

1) This side chain crystalline polycarbonate provides a high level of hydrophobic alkyl side chains to protect a water sensitive release material.
2) Side-chain crystalline polycarbonates from glycerin carbonate:

Glycerin carbonate has a free hydroxyl group which can be reacted with stearoyl anhydride to give the ethylene carbonate methylene crystalline fatty ester. Reaction of this intermediate with any diol, for example, 1,3-propanediol, leads to a crystalline fatty ester methylene polycarbonate diol:

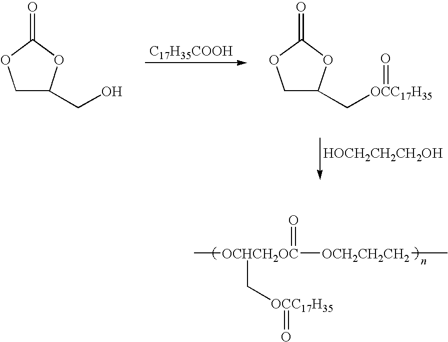

3) Side-chain crystalline polycarbonates from monoglycerides, dimethyl carbonate, and lactic/glycolic acid(s):

A monoglyceride obtained by mono-esterification of glycerin with a crystalline fatty acid, for example, stearic acid, gives a crystalline fatty monoglyceride which can be reacted with at least two moles or more of any combination of lactic, glycolic and 3-hydroxypropionic acids and then this polymeric diol can be reacted with dimethyl carbonate to yield a simple side chain crystalline polycarbonate:

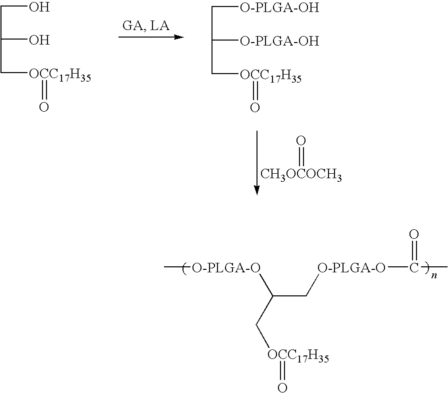

### CYSC Polyesters:

a. Side-Chain Crystalline Malic, Citric or Tartaric Acid Polyesters:

Malic, tartaric and citric acid contain one or more hydroxy groups. One of these hydroxy groups can be esterified with a crystalline fatty acid to give a polycarboxylic acid crystalline fatty ester which can be then reacted with a simple diol, 1,3-propanediol, for example, to provide a side chain crystalline polyester similar to the well known reaction of a polyol, trimethylol propane with a fatty acid and subsequently with a polycarboxylic acid to give a polyester backbone with pendant fatty acid ester groups. These kinds of polyesters have been used in the industrial coating business:

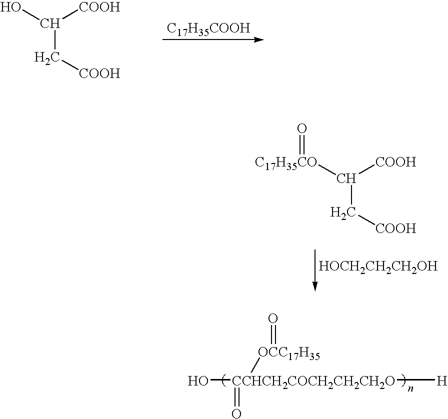

b. Side-Chain Crystalline Malic or Tartaric Acid PLGA Polyesters:

Tartaric acid is a di-acid having two hydroxyl groups—one adjacent to each of the carboxylic acid groups. Malic acid has one hydroxyl group adjacent to one of the two carboxylic acid groups. If tartaric or malic acid is esterified with a crystalline fatty acid, for example, stearic acid, a crystalline fatty modified polycarboxylic acid is made which can react with a simple diol, 1,3-propanediol, to give a side chain crystalline polyester as in a).

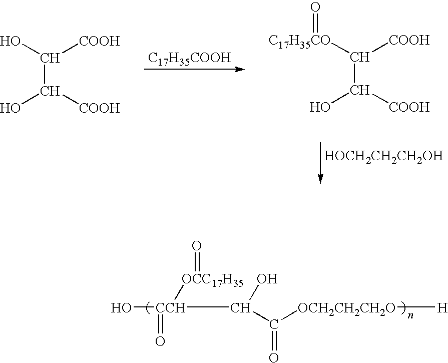

If on the other hand one of the acid groups of tartaric or malic acids is esterified with a crystalline fatty alcohol, a crystalline fatty ester hydroxyl acid is made. This product can then be reacted with stearic acid, stearyl alcohol or the like, to add one or two Cy moieties at the terminal units. This product can then be reacted with lactic and glycolic acids to give a side chain crystalline PLGA:

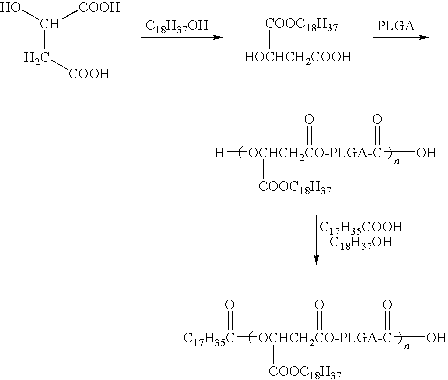

c. Polyesters from Glycidol, Crystalline Fatty Acid and Polybasic Acid:

Glycidol, an epoxy mono-alcohol, can be reacted in a one reaction vessel sequence in the presence of crystalline fatty acid and polycarboxylic acid, for example, succinic anhydride, to give a side chain crystalline polyester:

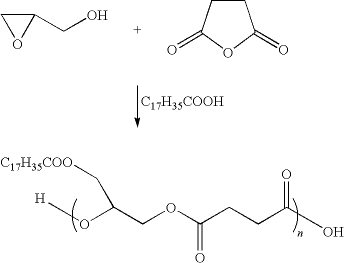

Or, the DMPA fatty crystalline alcohol ester can be reacted with a simple dicarboxylic acid (or precursor), for example, succinic anhydride, to yield a side chain crystalline polyester:

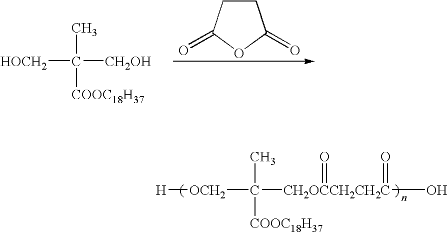

### A. Bioerodable Side-chain Crystalline Oxazolines:

TrisAmino, an amino polyol commercially available from Angus (Dow) and used in many medical applications where bioerodibility is critical can be first mixed and reacted with a fatty acid, for example, stearic acid, to give a fatty amide polyol which on heating cyclizes to a crystalline fatty oxazoline polyol. This polyol is easily reacted with a dibasic (or polybasic acid), in this case a dibasic acid precursor, succinic anhydride to give a side chain crystalline and bioerodable polyester-oxazoline:

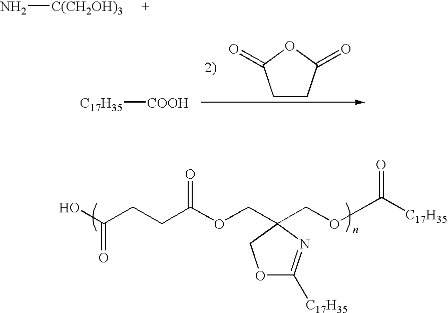

The oxazoline moiety may be desirable for mixing with some types of release materials to insure a uniform, compatible mixture of release material and polymer to provide repeatable and consistent release material delivery results with a low burst profile.

### B. Crystalline Alkyl Dioxanones:

A p-dioxanone can be prepared from a glycol by cyclizing with glycolic acid. When this intermediate is reacted with a simple diol, e.g. 1,3-propanediol or hexanediol, a copolymer of the 1,4-dioxane-2-one with the diol forms. Also, the 1,4-dioxane-2-one under heating and catalyst yields a homopolymer. If we use a long chain crystalline alpha-olefin oxide, and hydrolyze this olefin oxide to the crystalline long chain 1,2-dihydroxy alkane, we synthesize a crystalline substituted p-dioxanone. This crystalline dioxanone once polymerized will give a side chain crystalline polydioxanone:

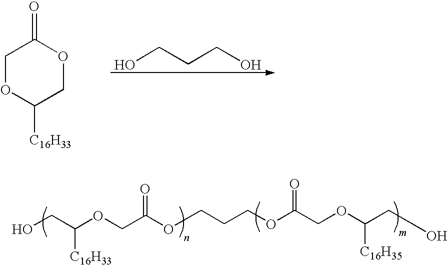

### ECC Polymers

In one embodiment, an ECC polymer comprises at least one moiety having the formula

-Qx(-b-Cy)_{q} (Q2)

wherein Qx is a moiety having a valence of at least (q+1), q is at least 2, for example 2, 3, 4 or 5, and b and Cy are as hereinbefore defined. The ECC polymer can for example contain two, three or four such moieties, which can be the same or different. The ECC optionally has one or more of the following characteristics:—
(1) it has a molecular weight of 3,000-20,000, e.g. 3,000-10,000 or 3,000-8,000;
(2) the backbone of the polymer consists essentially of carbon atoms, e.g. is a polyacrylate;
(3) the backbone of the polymer comprises bioerodable linkages, e.g. is a PGLA;
(4) the value of (Tp−To) is less than Tp^{0.7}, e.g. less than Tp^{0.6}, or less than 5° C.;
(5) the molar percentage of units comprising Cy moieties is less than 30% e.g. less than 20% or less than 10%, and/or more than 1%, e.g. more than 2% or more than 4%;
   in one embodiment, an ECC polymer has a backbone comprising bioerodable linkages, e.g. linkages having the formula

-D_{nd}-Q-Eₙₑ- (Q1)

as hereinbefore defined, and optionally has at least one of the following characteristics:—
(1) it has a molecular weight less than 10,000, e.g. less than 8,000, or less than 7,000, or less than 5,000, and/or a molecular weight greater than 600, e.g. greater than 800 or greater than 1000;
(2) the molar percentage of moieties comprising CYC moieties is less than 30% e.g. less than 20% or less than 10%, and/or more than 1%, e.g. more than 2% or more than 4%; and
(3) at least 50 mol percent, e.g. at least 70 mol % or at least 80 mol %, e.g. substantially all, of the repeating units forming the backbone of the polymer are free of Cy moieties;
   In one embodiment, an ECC polymer has a backbone which comprises (i) bioerodable linkages having the formula

—O—CO—O—

(ii) methylene moieties, and (iii) substituted methylene moieties in which the substituent is a b-Cy moiety, the polymer optionally having at least one of the following characteristics:
(1) it has a molecular weight less than 20,000, e.g. less than 15,000, or less than 10,000, or less than 8,000, or less than 5,000;
(2) the molar percentage of units comprising Cy moieties is less than 30% e.g. less than 20% or less than 10%, and/or more than 1%, e.g. more than 2% or more than 4%;
(3) it comprises Cy moieties which comprise an n-alkyl moiety containing 16-30, e.g. 18-22, carbon atoms;
(4) it has a value of (Tp−To) which is less than Tp^{0.7}, e.g. less than Tp^{0.6}, or less than 5° C.;
(5) it has a heat of fusion of 3-50, e.g. 3-30 or 3-22 or 3-10, J/g; and
(6) at least 50 mol percent, e.g. at least 70 mol % or at least 80 mol %, of the repeating units forming the backbone of the polymer are free of CYC moieties.

In one embodiment, an ECC polymer comprises repeating units having the formula

—R_{alkylene}—CO—O—

wherein R_{alkylene} is a substituted or unsubstituted straight chain or branched alkylene radical, for example —(CH₂)₂— or —CH(CH₃)—CH₂—; the CYC polymer optionally having one or more of the following characteristics:
(1) it has a molecular weight less than 10,000, e.g. less than 8,000, or less than 7,000, or less than 5,000, and/or a molecular weight greater than 500 or greater than 1000, e.g. greater than 1500, e.g. 2000-5000;
(2) the molar percentage of units comprising CYC moieties is less than 30% e.g. less than 20% or less than 10%, and/or more than 1%, e.g. more than 2% or more than 4%;
(3) the value of (Tp−To) is less than Tp^{0.7}, e.g. less than Tp^{0.6}, or less than 5° C.;
(4) at least some of the CYC moieties are present in terminal units having one or more of the formulas (J)-(L) below in which the R_{alkylene} moieties can be the same as or different from the R_{alkylene} moieties in the repeating units

—R_{alkylene}—CO—O-Cy (J)

—O—CO—R_{alkylene}—CO—O-Cy (K)

—O—CO—R_{alkylene}—CO—O—Yₜₑᵣₘ—R_{pbalc}—O-Cy (L)
where R_{pbalc} is the residue of a polyol, and has for example the formula

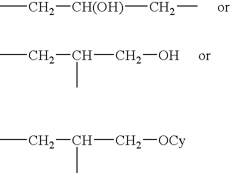

(5) the CYC polymer contains a plurality of polyalkylene ether units, for example a terminal polymeric block having the formula

HO—(CH₂—CH₂—O—)_{npeg}
where npeg is 2-150, e.g. 6-100;
(6) at least some of the CYC moieties are present in repeating units of formula (1) above, for example repeating units having the formula

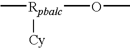

where R_{pbalc} is the residue of a polyol, for example a polyethylene glycol, 1,3-propanediol, glycerin or sorbitol, and optionally contains two or more CYC moieties;
(7) it contains repeating units having the formula

—R_{alkylene1}—CO—O— and
repeating units having the formula

—R_{alkylene2}—CO—O—
wherein -R_{alkylene1}- is different from -R_{alkylene2}-, e.g. one is —(CH₂)₂— and the other is —CH(CH₃)—CH₂—, and
(7a) at least one of —R_{alkylene1}—CO—O— and —R_{alkylene2}—CO—O— is present only in the form of moieties having the formula

(—R_{alkylene1}—CO—O—)_{N}

or

(—R_{alkylene2}—CO—O—)_{N}
respectively, wherein N is equal to (1+X) wherein X is zero or an even integer, or
(7b) at least some of the units are not derived from a cyclic dimer, or
(7c) at least one of —R_{alkylene1}—CO—O— and —R_{alkylene2}—CO—O— is not present only in moieties having the formula

(—R_{alkylene1}—CO—O—)₂

or

(—R_{alkylene2}—CO—O—)₂
respectively;
(9) it has a heat of fusion of 3-50, e.g. 3-30 or 3-22 or 3-10, J/g
(10) at least 50 mol percent, e.g. at least 70 mol % or at least 80 mol %, of the repeating units forming the backbone of the polymer are free of CYC moieties.

Preferred ECC polymers for use in this invention
(A) comprise a plurality of polymeric molecules each of which consists essentially of
   (i) a polymer backbone which comprises a plurality of repeating units having the formula

—CF¹F²—CO—O— (ECC 1)
wherein
F¹ is hydrogen and F² is hydrogen or methyl, the repeating units being the same or different, and
(ii) at least one terminal unit which has the formula

-b-Cy (ECC 2)
wherein
Cy is an n-alkyl moiety containing 18-50, e.g. 18-24, carbon atoms, and
b is a bond or a moiety which has a valence of at least 2 and which links the Cy moiety to the polymer backbone, and which optionally contains one or more additional Cy moieties;
(B) has a Tp of at least 40° C., a To such that the value of (Tp−To) is less than Tp^{0.7} and a ΔH of at least 5 J/g, and
(C) has an Mn of less than 10,000.

In such ECC polymers containing more than one terminal unit of formula (2), and/or a terminal unit containing two or more Cy moieties, for example a total of 2, 3, 4 or 5 Cy moieties in one or more terminal units of formula (2), one or both of b and Cy can be the same or different in the different terminal units, and in a terminal unit containing more than one Cy moiety, the Cy moieties can be the same or different. The ECC polymer can optionally contain, in addition to the units of formulas (ECC 1) and (ECC 2), repeating units and/or terminal units having a different formula. For example, the repeating units can be derived from a mixture of lactic acid and glycolic acid, and the polymer can contain two terminal units of formula (2), each containing at least one Cy moiety, e.g. an n-alkyl moiety containing 18 carbon atoms.

These preferred ECC polymers can for example be prepared by endcapping a preformed PLGA by (i) reaction of the carboxyl end group of the PLGA with an alcohol containing a Cy moiety, e.g. stearyl alcohol or behenyl alcohol, or (ii) reaction of the carboxyl end group of the PLGA with a polyhydroxy compound, e.g. a sugar such as sorbitol, followed by esterification of one or more of the remaining hydroxy groups with a carboxylic acid (or the like) containing a Cy moiety, and/or (iii) by reaction of the hydroxyl end group of the PLGA with an n-alkyl carboxylic acid (or the like) in which the n-alkyl moiety contains 18-50, e.g. 18-22, carbon atoms.

These preferred ECC polymers can consist essentially of the moieties of the formulas (ECC 1) and (ECC 2), i.e. the polymer is derived from a PGA (in which each of F¹ and F² is hydrogen in each of the repeating units), a PLGA (in which each of F¹ and F² is hydrogen in some of the repeating units, and one of F¹ and F² is hydrogen and the other of F¹ and F² is methyl in the other repeating units) or a PLA (in which one of F1 and F2 is methyl and the other of F1 and F2 is hydrogen in each of the repeating units). Alternatively, the polymer can also contain repeating units of a different type, optionally providing further carboxy linkages in the polymer backbone, for example units derived from caprolactone. The following statements disclose optional characteristics of these preferred ECC polymers. Two or more of the stated characteristics can be present at the same time, except where the context makes this impossible.

(A) the polymer
(A1) has a molecular weight less than 8,000, or less than 7,000, or less than 5,000;
(A2) has a value of (Tp−To) which is less than Tp^{0.7}, e.g. less than Tp^{0.6}, or less than 10° C.;
(A3) contains less than 30%, e.g. less than 20% or less than 10%, and/or more than 1%, e.g. more than 2% or more than 4%, molar percent of units comprising Cy moieties;
(A4) has a heat of fusion of at least 10, or at least 20 J/g;
(A5) at least 50 mol percent, e.g. at least 70 mol % or at least 80 mol %, of the repeating units forming the backbone of the polymer are free of Cy moieties;
(A6) the terminal units have one or more of the formulas (2A)-(2C) below

-Cy (2A)

—CO—O-Cy (2B)

—R_{pbalc}—CO—Cy (2C)
where R_{pbalc} is the residue of a polyol, for example a polyethylene glycol, 1,3-propanediol, glycerin or sorbitol, and optionally contains one or more Cy moieties, the terminal and other Cy moieties, if any, being introduced for example by esterification, transesterification or alkylation of one or more of the hydroxyl groups, e.g. by reaction with an acid or acid chloride containing a Cy moiety; and has for example the formula

—CH₂—CH(OH)—CH₂—O—CO-Cy

or —CH₂—CH(O—CO-Cy)—CH₂—O—CO-Cy;
(A7) contains less than 170 repeating units of formula

—CF¹F²—CO—O—;
(A8) is prepared by a process which comprises copolymerizing one or more monomers, i.e. one or more of lactic acid and glycolic acid, and their cyclic dimers, lactide and glycolide, which will result in the repeating units of formula (1) and one or more monomers or components which will result in the terminal units which have formula (2) or which can be converted into terminal units of formula (2). For example, (i) an alcohol containing a Cy moiety can be reacted with lactic acid and glycolic acid, or (ii) a polyol, for example glycerine, 1,3-propanediol or 1,6-hexanediol, can be reacted with glycolic acid and lactic acid to form a PLGA glyceride ester which is end capped with an excess of a carboxylic acid containing a Cy moiety, for example stearic acid or behenic acid;
(A9) is prepared by endcapping a preformed polymer having the formula HO—(—CF¹F²—CO—O—)ₙ—H
where n is an integer less than 170; the preformed polymer, if prepared by the polymerization of lactide and glycolide, will consist predominantly of pairs of identical repeating units, and if prepared by the polymerization of the monomers (lactic and glycolic acids) will have randomly distributed repeating units; for example
   (A9a) is prepared by a process which includes the step of reacting the terminal hydroxyl group of a preformed PLA, PLGA or PGA polymer with a monomer or component, e.g. a carboxylic acid or acid chloride which contains a Cy moiety, or with a monomer or component which contains a moiety which can be further reacted so that it comprises a Cy moiety; or
   (A9b) the polymer is prepared by a process which includes the step of reacting the terminal carboxyl group of a preformed PLA, PLGA or PGA polymer with a component, e.g. an alcohol, which contains a Cy moiety, or with a component which contains a moiety which can be further reacted, e.g. by transesterification, so that it comprises a Cy moiety. For example, the preformed polymer can be reacted with (i) an alcohol containing a Cy moiety, or (ii) a polyol, for example glycerine, 1,3-propanediol or hexanediol, followed by reaction of one or more of the remaining hydroxyl groups of the polyol with an excess of a carboxylic acid containing a Cy moiety, for example stearic acid or behenic acid; or
   (A9c) is prepared by a process which includes the step of reacting the terminal hydroxyl group of a preformed PLA, PLGA or PGA polymer with a dicarboxylic acid or anhydride, e.g. succinic anhydride or succinic acid (succinylation); the resulting product, which may have a relatively high molecular weight in which case it may be in the form of a gel, facilitates formulation mixing in-situ, and/or may enable easy mixing of a protein or peptide release material at one pH and release at another pH; or
   (A9d) is prepared by reacting a preformed PLA, PLGA or PGA polymer with methacrylic anhydride and this reaction mixture is copolymerized with an ethylenically unsaturated monomer containing a Cy moiety; or
   (A10) contains at least 10%, e.g. 10-40%, by weight of the Cy units.

### Other ECC Polymers

A polyol reacted in the presence of lactic acid and glycolic acid forms PLGA sequences with OH end groups at each hydroxyl group of the polyol. These terminal hydroxyl groups can be end capped with a crystalline fatty acid, for example, behenic acid:

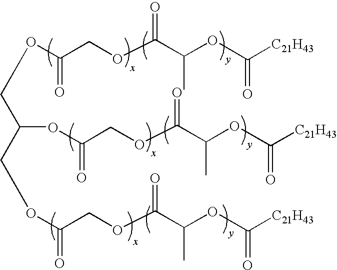

The polymer formed by reaction of a polyol with PLGA monomers (lactic acid+glycolic acid) and end-capping with a crystalline fatty acid gives a fatty acid ester of a hydroxyl PLGA glycerate.

Alternatively, a hydroxyl polycarboxylic acid like citric acid is esterified with a crystalline fatty acid (stearic acid) at the sole remaining hydroxyl group to provide a citric acid stearate. This intermediate is subsequently reacted with lactic acid and glycolic acid to give an acid terminated tri-PLGA citrate which may be end-capped with crystalline fatty alcohol:

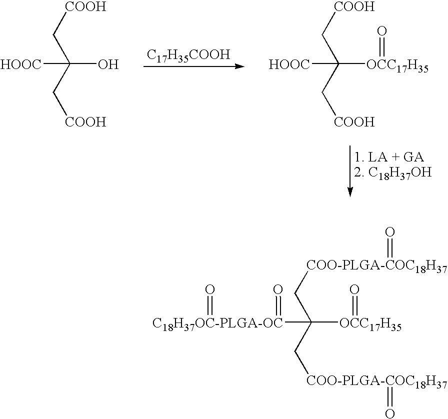

d. End-Cap Crystalline Glycerin Carbonate ECC-PLGA Polyesters:

Glycerin carbonate is easily mixed and reacted with a crystalline fatty acid, for example, stearic acid, to yield a disubstituted hydroxymethyl ethylene glycol diester. This intermediate is then polymerized with lactic acid and glycolic acid or esterified with a preformed PLGA and the larger molecule then optionally end-capped:

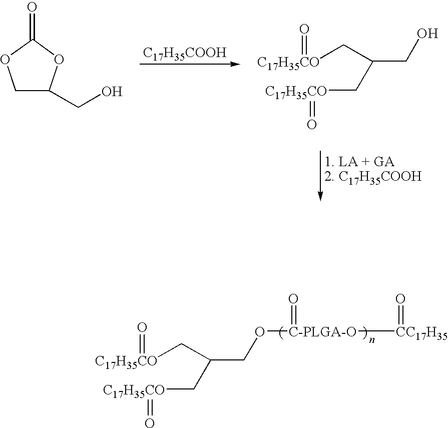

The amount of end capping with fatty acid is dependent on the degree of hydrophobicity and crystallinity desired in the final product. This will be dependent upon the release material which is mixed with the polymer, the release material solubility parameter compared to the polymer solubility parameter and the desired controlled release properties. In general the higher the crystallinity the longer will be the release and the slower will be the bioerodibility. Also, the higher crystallinity will exhibit a lower burst effect than a very low amount of crystalline fatty acid end capping.
e. ECC-12-hydroxystearic acid PLGA Polyesters:

12-hydroxystearic acid, readily available from castor oil, can be polymerized with lactic acid and glycolic acid and end capped with fatty acid to provide a multi-block PLGA with a hydrophobic center and two end cap crystalline groups for mixing with small molecule insoluble release materials for a slow controlled release release material delivery vehicle:

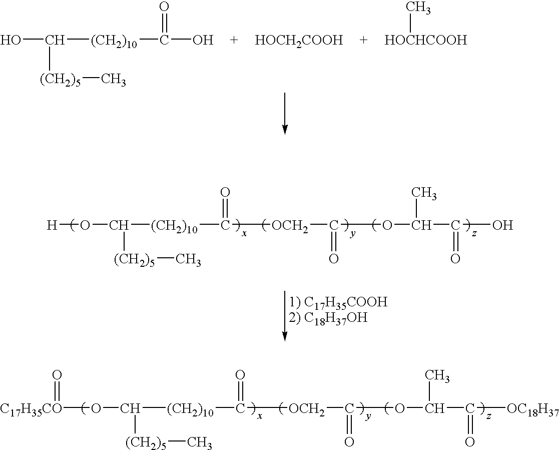

f. Crystalline Fatty Ester Dimethylol Propionic Acid Polyesters:

Dimethylol propionic acid (DMPA) can be esterified with a crystalline fatty alcohol and the resulting crystalline fatty ester polyol reacted with either (a) PLGA components and end-capped with crystalline fatty acid or (b) alkyl functional PLGA to give a novel end cap PLGA with a central and two terminal crystalline, hydrophobic blocks -An A-B-A-B-A block where the A components are hydrophobic and the B component is less hydrophobic and in some cases hydrophilic:

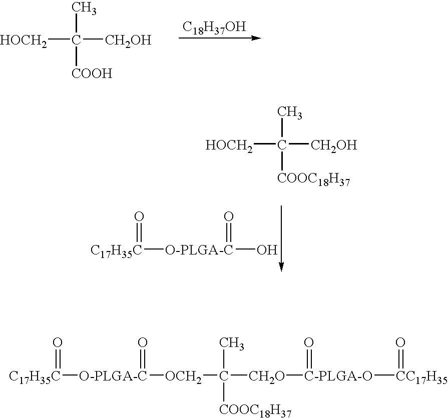

This kind of CYC polymer by its structure and molecular weight can have different levels of crystallinity—both side chain and end capped to provide easy release material mixing and loading and the desired sustained release properties.

In a similar manner, the preformed polyol may be reacted with lactic acid and glycolic acid to give a crystalline oxazoline PLGA polyester which can be end capped with more crystalline fatty acid if desired:

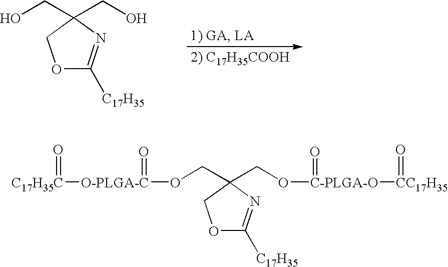

### C. Side-Chain Crystalline Hyaluronic Acid:

A dilute hyaluronic acid (HA) solution can transport a CYSC polymer mixed with a release ma is. She is terial and suspended in the hyaluronic acid solution as a finely dispersed suspension. If the HA is esterified by reacting one or two free hydroxyl groups on the HA, then a side chain crystalline HA is formed which may function as a CYC carrier, the Cy groups providing a way to regulate dissolution of cosmetic material from the normally highly water soluble HA solution:

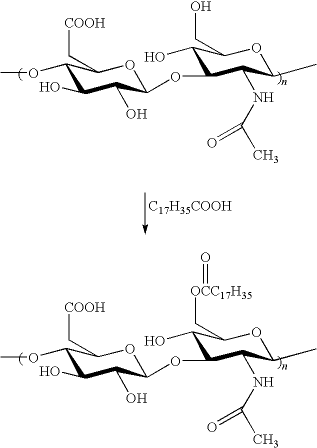

### CYC Assemblies

In CYC assemblies, crystalline character is created or enhanced by the non-covalent assembly (“self-assembly”) of complementary units in different molecules. Similar self-assembly takes place in nature, for example in peptide architectures and the replication of DNA. The self-assembly can be the result only of non-covalent and non-ionic interaction of complementary units. However, ionic bonding can also be involved. For example an acid-terminated PLGA can be mixed with a crystalline difatty nitrogen amine (NR₂H, where R comprises a Cy moiety) to provide an ionic salt which is crystalline as evidenced by DSC but which does not contain a covalent bond.

Any of the CYC carriers can take part in self-assembly, either with another CYC carrier or with another monomer or polymer which includes complementary moieties which will “self assemble” with moieties in the CYC carrier. An entity which is not itself a CYC carrier and which includes the complementary moieties is referred to herein as a “self-assembly additive”. Two or more self-assembly additives, neither or none of which is a CYC carrier itself, can form a CYC assembly. The self-assembly results in a product which has a higher ΔH (as compared to the calculated ΔH) and which may have a different Tp than the CYC carrier or the self-assembly additive itself. Because the self-assembly results in a higher ΔH and can result in a higher Tp, the CYC assemblies can make use of components which are similar to the CYC carriers but which do not meet either or both of the requirements that the Tp is at least 0° C. and the ΔH is at least 3 J/g, provided that the CYC assembly does meet those components. The self-assembly can also result in a significant difference in the rate at which a release compound is released from a release composition.

The presence of a self-assembly additive can assist in extending release of a release material. In many cases, the self-assembly additive will be slowly released from a release composition.

In one embodiment, the CYC assembly comprises two or more of a CYSC polymer, a CYSC compound and an ECC polymer. The self-assembly can be between the Cy moieties and/or through other complementary moieties, as described below. For example, the CYC assembly can comprise an endcapped PLGA copolymer and a CYSC acrylate polymer. Mixing as little as 3% of a CYSC acrylate polymer with an endcapped crystalline PLGA can result in a significantly different release rate.

The complementary moieties in a self-assembly additive can be Cy moieties which self assemble with Cy moieties in the CYC carrier, e.g. n-alkyl moieties containing 14-50, preferably 16-24, e.g. 18 or 22 carbon atoms. Alternatively or additionally, the complementary moieties can be other moieties which self assemble with similar moieties in the CYC carrier, for example polyoxyethylene or other polyoxyalkylene moieties, for example containing, 240, 2-20 or 2-10 oxyalkylene moieties. The self-assembly additive can also contain functional groups, e.g. OH, COOH, CONH₂ and CONR₂. The complementary moieties, and the functional groups if present, can for example be present in alcohols, ethers, acids or derivatives of acids, for example esters or amides. Examples of self-assembly additives include stearic acid, behenic acid, palmitic acid, stearyl alcohol, behenyl alcohol, hexadecyl alcohol, stearamide, N,N,-dimethyl stearamide and the like. Examples of self-assemblies included block type structures like polyoxyethylene stearyl alcohol (PEO6 stearyl alcohol) and other block coploymer analogs. For example, a CYC additive (which may also be a CYC carrier) can contain units derived from an amphiphilic monomer and a pegylated or ethyleneoxy-containing side chain (e.g. alkoxypolyoxyethylenemethacrylate where the alkoxy group is C1 to C22 and the number of oxyethylene groups is from 2 to 24) can be blended with a self-assembly additive which is a crystalline alkyl polyoxyethylene alcohol wherein the number of oxyethylene units are from 2 to 40, preferably 2 to 20, and more preferably 2 to 10.

Self-assembly can increase Tp and ΔH by a greater degree than predicted by a simple “rule of mixtures”, thus confirming that co-crystallization occurs. It is also possible that self-assembly results in more of the Cy moieties becoming part of crystalline domains. The presence of a self-assembly additive can influence the rate of release and can also result in a more biodegradable formulation than a formulation composed of the release material and CYSC polymer alone.

For example, a fatty acid, e.g. stearic acid, may be mixed with an end cap fatty crystalline PLA, PGA or PLGA copolyme to give a crystalline material which has effective capability in the sustained release of a release material in the formulation. For example, a crystalline fatty ether alcohol (e.g. a stearyl polyoxyethylene alcohol containing two moles of EO functionality) can form a non-covalent CYC carrier with enhanced crystallinity when mixed with a crystalline CYSC or end-cap crystalline bioerodable polymer.

### Preparation of CYC Carriers

The CYC carriers can be prepared in any way, for example using techniques which are well-known to those skilled in the art. For example, the CYC polymers can be prepared by emulsion, solution, bulk and suspension polymerization techniques using conventional catalysts. Conventional additives and catalysts can be employed to achieve desired molecular weights. For example, in the preparation of CYSC polymers, azo and peroxide catalysts, thiol chain transfer agents (e.g. alkyl mercaptans, hydroxyethyl mercaptan, butyl mercaptopropionate and mercapto acetic acid), or allyl chain transfer agents or regulators (e.g. including alpha-methyl styrene) can be used. The type of polymerisation can often be selected according to the form of CYC release composition to be administered. For example, if a micelle or emulsion form is desired, emulsion polymerisation, optionally in the presence of the release material, can be employed. If a hydrogel is preferred, polymerisation under aqueous or emulsion conditions can be employed. If a spray-dried form is preferred, polymerisation under solvent conditions can be used.

Methods of preparing CYSC graft copolymers include for example preparing a preformed polymer comprising Y(Rc) moieties and optionally Z(Rz) moieties, and then grafting suitable monomers (which may contain Rc and/or Rz moieties) at reactive sites at the end or in the middle of the preformed polymer. Methods of preparing CYSC block copolymers include preparing two or more preformed polymers, at least one of the preformed polymers comprising Y(Rc) moieties and optionally Z(Rz) moieties, and at least one of the other preformed polymer(s) comprising Z(Rz) moieties, each of the preformed polymers having at least one reactive site at an end of, or between the ends of, the preformed polymer, and then reacting the preformed polymers to form the desired polymer.

For example, a CYSC block polymer can be prepared by copolymerising a vinyl type macromonomer with other monomers, or by making a CYSC polymer, and then reacting the functionalized polymer with the second block material, for example a urethane block, or an epoxy block, a polyether block, a polyester block, a polyethyleneoxide, polypropyleneoxide or polytetramethyleneoxide block, a polysiloxane block, or a poly(alkyl or alkoxy)silane block.

### Specific Exclusions and Conditions

Certain isolated embodiments of the invention exclude the possibility that the CYC polymer is a CYSC polymer which has one or more of the following characteristics

(1) the polymer is a block co-polymer or a graft co-polymer;
(2) the polymer is a hydrogel;
(3) the polymer is not hydrolytically stable;
(4) the polymer contains moieties derived from a vinyl amide;
(5) the polymer is cross-linked;
(6) the polymer is a thermoplastic elastomer;
(7) the “b” moiety is an anhydride;
(8) the polymer contains 15-20 mol % of methacrylic acid units;
(9) the polymer contains 20-40 mol % of methacrylic acid units;
(10) the polymer contains 15-20 mol % of acrylic acid or alkyl acrylic acid units;
(11) the polymer contains 20-40 mol % of acrylic acid or alkyl acrylic acid units;
(12) the polymer contains side chains containing 18 carbon atoms;
(13) the polymer contains side chains containing 12-18 carbon atoms;
(14) the polymer contains a carbon atom which is directly linked to the Cy moiety and also to a moiety containing a carboxyl or carboxyl salt moiety;
(15) the polymer is in the form of a film;
(16) the polymer is a random copolymer;
(17) the polymer is an elastomer;
(18) the polymer contains a CYC moiety which is attached to the polymer backbone through an anhydride linkage;
(19) the polymer contains a CYC moiety which contains anhydride linkages;
(20) the polymer contains the structure Ra—CO—O—CO—Rb where Ra and Rb may be any moiety;
(21) the polymer does not contain more than 40% of units containing the Cy moieties;
(22) the polymer contains a polybasic acid;
(23) the polymer does not have a pKa greater than 4;
(24) the polymer is used as a gating membrane which is placed between an interior enclosure comprising the release material and an exterior volume into which the release material is to be dispensed;
(25) the polymer is cross-linked or otherwise rendered nonflowable, e.g. by making it part of a block copolymer containing a high melting block, or immobilizing it within a microporous membrane, hollow fiber or fabric mesh;
(26) the polymer is prepared by emulsion polymerization;
(27) the polymer does not contain biodegradable linkages other than ether and ester linkages between repeating units in the polymer backbone, and/or biodegradable linkages other than ester and amide linkages between the polymer backbone and Cy.

### Release Compositions, Methods of Making Release Compositions and Methods of Delivering Release Materials

The release compositions of the invention comprise a CYC carrier and a release material associated therewith. In some embodiments, the release material is dissolved in the CYC carrier, thus forming a single phase. In other embodiments, the release material is uniformly or non-uniformly distributed as a separate phase in the CYC carrier, for example as a dispersion or emulsion. In some embodiments, the release composition is a single phase composition. In other embodiments, the release composition comprises at least two phases, for example comprising a matrix in which the CYC carrier and release material are uniformly or non-uniformly distributed as a separate phase (which, as indicated above, can comprise a solution of the release material in the CYC carrier or a uniform or non-uniform distribution of the release material as a separate phase in the CYC carrier).

The Tp of a release composition can be the same as the Tp of the CYC carrier, but is more often somewhat lower than the Tp of the CYC carrier. The Tp of a release composition can for example be 22-70, 35-50, 38-50, 37-42, or 40-47° C., or at least 37° C.

The composition can contain any appropriate proportion of the release material, depending on the intended use of the composition. In some embodiments, the weight of the release material, based on weight of the composition is at least 1%, at least 2%, at least 5%, at least 7%, at least 8%, at least 10%, at least 15%, at least 17%, at least 20%, at least 25%, at least 30%, at least 35%, at least 40%, at least 45%, at least 50%, at least 55%, at least 60%, or at least 70%, e.g. 1-20%, 5-30%, 10-30%, 20-30%, 10-50%, or 20-50%. In other embodiments, the composition contains 0.1-5%, for example up to 2%, up to 3%, or up to 4%, of the release material.

The CYC carrier should be selected having regard to the nature of the release material, the way in which the release composition is to be applied to the target site, and the way in which the release material is to be released.

A simple form of association is a solution of the release material in the CYC carrier. Another form is a dispersion, an emulsion or a suspension in which particles comprising the release material and the CYC carrier are uniformly distributed in an aqueous medium.

In some embodiments, the release composition comprises a colloidal dispersion of particles having a size of 1 nm to 0.5 mm. Such a dispersion can be produced by mixing or sonication or homogenization of an aqueous mixture of the release material and polymer matrix in the presence of a surfactant. The release material and/or the CYC carrier may be dissolved in a solvent which evaporates during the sonication or homogenization process. This process can lead to microparticles having a size of from 0.1 to 1000 microns. These colloidal and emulsion mixtures may be suitable for a variety of applications. For example, microsphere particles may have a diameter of 0.1-150, e.g. about 50, μm, measured using a Horiba particle size analyzer LA 910.

The release material may influence the processing conditions. For example, some release materials are likely to be adversely affected by conventional emulsification used for microsphere production.

In one method for making compositions comprising a CYC carrier and a release material, the CYC carrier is melted, and the release material is mixed with the CYC carrier, the mixing being carried out at a temperature which is above the To, usually above the Tp, of the CYC carrier. During such mixing, other desired ingredients, e.g. fillers, excipients, dyes, colorings, flavors, disintegrators, stabilizers, can be added. The mixing can optionally be carried out in the presence of another material which is liquid at the mixing temperature and which is not a CYC carrier. This method can result in a uniform mixture which can, if desired, be suspended above the melting point of the mixture in a non-solvent, thus producing, upon cooling, solidification of the release composition as particles. The particles can be washed and filtered, and, for example, suspended in a liquid carrier. In another embodiment, in which no solvent is preferably used, the molten mixture, or the solidified mass obtained by cooling the molten mixture, can be processed into desired shapes, e.g. into rods, ovals, and tablets, using known procedures.

Because the CYC carrier can have a relatively low melting point, the ability to carry out mixing at relatively low temperatures is particularly valuable when the release material is damaged by exposure to a temperature which is more than 15° C., or more than 30° C., above the Tp of the CYC carrier, for example more than 50° C. or more than 65° C. The CYC carrier used in such methods may for example have a Tp about (or alternatively not more than) 30° C., 35° C., 37° C., 40° C., 42° C., 45° C., 50° C., 55° C., 60° C., 65° C., 70° C., 75° C. or 80° C., for example 35-65° C., 40-65° C., 45-60° C., 40-60° C., 50-65° C. In some cases, it is desirable that the formulation should have a Tp above the body temperature of the subject (e.g. 37° C.) However, in some embodiments, the formulation has a Tp below 37° C.

In another embodiment, microparticles are prepared by dissolving a CYC carrier in a solvent, dispersing a solid or liquid release material into the solution, and dispersing the mixture rapidly into cold water or spray in the mixture into a chamber, thus forming microspheres or microcapsules.

Another method of making microparticles is the water/oil/water double emulsion method. An aqueous phase containing the release material, is dispersed into an oil phase comprising the CYC carrier dissolved in an organic solvent under high speed homogenization conditions. The resulting water-in-oil emulsion is then dispersed in an aqueous solution containing a polymeric surfactant such as a polyvinyl alcohol and further homogenized to produce a wa ter/oil/water emulsion. The emulsion is stirred for several hours to evaporate the solvent, and the resulting nanoparticles or microparticles are collected by filtration.

In another embodiment, a CYC polymer is prepared as an emulsion copolymer using emulsion polymerization techniques, optionally in the presence of small amounts of co-solvent. The release material can then be added in a compatible solvent for the emulsion, thus allowing migration of the release material into the emulsion polymer particles.

The CYC carrier can be used in combination with other polymers, for example amorphous polymers. For example, the release composition can also contain simple acrylate copolymers which are good film formers, particularly where film-forming properties in addition to delivery properties are important.

The presence of acid groups in the CYC carrier may also make it possible to create a fine suspension of the release material/CYC carrier matrix in a suitable aqueous medium.

In some embodiments, the CYC carrier, preferably, a CYSC acrylate or ECC PLGA, has a Tp above the normal body temperature of the human body, so that when the composition is exposed to external heating, the release material is released.

In various embodiments, the CYC polymer is mixed with one or more of the following:—
(1) Synthetic or naturally occurring polymers (hydrophobic or hydrophilic), for example PLGA, poly(ethylene glycol), poly(ethylene oxide), polypropylene oxide, polypropylene glycol, poly(vinyl alcohol), polyurethane, collagen, gelatin, chitosan, sugar, and various derivatives of cellulose, etc.
(2) an additive that will self assemble (co-crystallize) with the CYC polymer, for example a fatty acid, a fatty alcohol, or an alkylated PEG, optionally containing the same or different Cy moieties. Suitable additives include ingredients sometimes used in personal care compositions, e.g. stearic esters, stearic acid, stearyl alcohol and ethoxylated stearyl alcohol. The self-assembly can be advantageous in storage of the formulation and application of the formulation. For example, if the Tp of the polymer is close to skin temperature, application of the formulation to the skin can provide a valuable sensation and valuable slip-on qualities, allowing for rapid application of the formulation as a thin uniform layer
(3) Excipients, including, but not limited to carbon nanotubes, hydroxyapatite and other additives, preferably biodegradable additives, that can improve the mechanical properties of the polymer composition.
(4) Emollients, for example oils and fatty acids, particularly in hand wash or other products which typically contain ethanol or another alcohol, which tends to wash away skin oils, and which optionally contain antimicrobial agents; in such mixtures, the CYC carrier can function as a thickener and/or simply a carrier of an emollient.
(5) Cosmaceuticals or nutraceutical materials which are preferably in contact with skin for an extended period, since such materials can be combined with CYC carriers which release the material over an extended period, e.g. at least 5 or at least 30 minutes, or at least 2 hours. For example, the CYC carrier can be part of a dermatological product or hair care product which delivers a bioactive compound to the skin or hair, for example a peroxide or other oxidizing agent, e.g. for the treatment of acne or another skin condition.

### SSP Polymers

In the SSP polymers, the moieties Y_{ch}, b and Cy can be of any kind (within the definitions given). In SSP polymers containing different moieties of formula (1), the moieties can differ from each other in one or more of Ych, b and Cy. A wide variety of Y_{ch}, b and Cy moieties is described above. The moieties Z and Rzphil can be of any kind. In SSP polymers containing different moieties of formula (2zphil), the moieties can differ from each other in one or both of Z and Rzphil. A wide variety of suitable units of formula (2zphil) is described above, including, for example, units derived or derivable from one or more of acrylic acid, methacrylic acid, acrylamide, methacrylamide, AMPS (2-acrylamido-2-methylpropane sulfonic acid), acrylonitrile, methacrylonitrile, and other oxygen-containing and nitrogen-containing monomers.

When Rzphil is an acid group, for example, a carboxyl group, this provides the CYC carrier with functionality available for pH modification, thus altering the solubility properties of the CYC carrier. For example, a CYC carrier containing acid groups can be more easily removed with soap or a shampoo.

The moieties of the formulas (1) and (2zphil) can be derived from monomers which are randomly copolymerized together. However, the fact that the molar ratio of the units of formula (2zphil) to the units of formula (1) is at least 2.5:1 ensures that there will be at least some blocks containing a plurality of (2zphil) units. Alternatively, the polymerization can be carried out in a way that there are distinct blocks of each type of unit.

An SSP polymer will of course contain terminal units having a formula different from formulas (1) and (2zphil). The terminal units can for example be terminal units derived from one or more of the monomers from which the repeating units of formulas (1) and (2zphil) are derived, or modifications of such terminal units. In addition, an SSP polymer can optionally contain, in addition to the repeating units of formula (1) and (2zphil), and the terminal units, repeating units having a different formula. Purely by way of example, Y_{ch} can be a

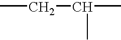

moiety, b can be a —CO₂— moiety and Cy can be an n-alkyl moiety containing 16-30 carbon atoms, for example derived from stearyl or behenyl acrylate or other n-alkyl acrylate or methacrylate; and the (2zphil) unit can be a moiety, derived from acrylic acid.

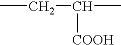

Known crystalline polymers whose crystallinity derives from long chain alkyl side chains are opaque. Surprisingly, however, some of the SSP polymers are transparent when solid. Such polymers are referred to herein as nanocrystalline polymers. The term “nanocrystalline polymer” is used herein to denote a polymer which, when formed into a thin film and tested by ASTM D 1003, has a haze value less than (preferably less than 0.8 times or 0.5 times) the haze value of a thin film which is composed of a copolymer of C18A and acrylic acid in a 1:1 molar ratio and which is formed and tested in the same way. The tested film exhibits a first order melt transition when measured by a DSC on the first heating cycle. The inventors believe that the crystal size of the nanocrystalline polymers is smaller than is required to scatter visible light, probably less than 100 nanometers. The parameters influencing the formation of a clear crystalline polymer are not only related to the co-monomers, chemical composition, molecular weights and viscosity, but are also influenced by the kinetic process of crystallization. Those skilled in the art will have no difficulty, having regard to their own knowledge and the disclosure of this specification, in determining whether or not a particular SSP polymer is a nanocrystalline polymer, and if it is not, what changes should be made to the polymer in order to increase the likelihood of obtaining a nanocrystalline polymer.

The SSP polymers of the present invention can optionally have at least one of the following characteristics.

(1) The molecular weight (Mn) of the polymer (measured as hereinbefore described) is at least 2000, e.g. at least 3,000, and/or at most 100,000, e.g. at most 50,000, or at most 25,000, or at most 12,000, particularly at most 10,000, e.g. 3000 to 12,000, or 3000 to 8000, or 4000 to 6000, if a nanocrystalline polymer is desired.
(2) The polymer is a polyacrylate.
(3) The repeating units of formula (1) comprise a straight chain polyalkylene moiety containing 17-21 —CH₂— moieties, for example derived from C18A and/or C22A.
(4) The polymer has a Tp of 40-80° C., preferably 42-63° C.
(5) The polymer has a Tp which is not more than 12° C. lower than, preferably not more than about 10° C. lower than, the Tp of a polymer which consists essentially of units of the same formula (1).
(6) The polymer has a (Tp−To) value of less than 10° C., preferably less than 8° C., for example 5-8° C., e.g. about 6° C.
(7) All the Ych and Z moieties are the same.
(8) The Rzphil moiety preferably has an Fₚᵢ value greater than 300 J^{1/2}cm^{3/2} mol⁻¹ highly polar (for example —COOH and —CN moieties have Fₚᵢ values of 420 and 1100). Examples of suitable Rzphil moieties include —COOH, —SO₃H, —CONH₂, six law —CONHR and —CONR₂, where R is for example lower alkyl, and —CN.
(9) The Rzphil moieties contain less than three carbon atoms, preferably 1 or 2 carbon atoms.
(10) The units of formula (1) consist essentially of units of the formula

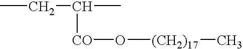

the units of formula (2zphil) consist essentially of units of the formula

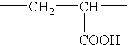

the molar ratio of the units of formula (2zphil) to the units of formula (1) is 2.5:1 to 4.5:1, e.g. 2.8:1 to 4.2:1; and the Mn of the polymer is preferably less than 7,000, particularly about 5,000.
(11) The units of formula (1) consist essentially of units of the formula

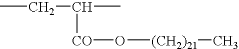

the units of formula (2zphil) consist essentially of units of the formula

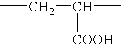

the molar ratio of the units of formula (2zphil) to the units of formula (1) is 3.5:1 to 4.5:1, e.g. 2.8:1 to 4.2:1; and the Mn of the polymer is preferably less than 7,000, particularly about 5,000.
(12) The units of formula (1) consist essentially of a mixture of units of the formula

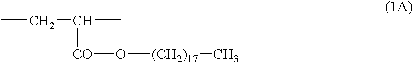

and units of the formula

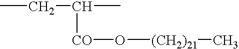

the molar ratio of the units of formula (1B) to the units of formula (1A) is 2.1:1 to 2.5:1; the units of formula (2zphil) consist essentially of units of the formula

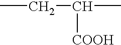

the molar ratio of the units of formula (2zphil) to the units of formula (1) is 2:1 to 3:1, e.g. 2.2:1 to 2.6:1; and the Mn of the polymer is preferably less than 7,000, particularly about 5,000.

The normal effect of large amounts of added comonomer in a CYSC polymer is to significantly reduce Tp, to significantly increase (Tp−To) and to significantly reduce the heat of fusion, because there are not as many side chain crystalline groups to develop large crystalline domains in below Tp. Surprisingly, in the nanocrystalline copolymers of this invention, this does not happen, in spite of the fact that the molar ratio of the units of formula (2zphil) to the units of formula (1) is more than 3, e.g. 3-6. Thus, the melting point is depressed only by a modest amount—about 10° C. or less—from the melting point of the same polymer without the units of formula (2zphil). It would have been expected that the Tp would have been depressed by much more than 10° C. at such molar ratios. In addition, the melting range of the nanocrystalline copolymer is about as sharp as the same polymer without the units of formula (2zphil).

It appears that in the nanocrystalline polymers, the Cy moieties crystallize together in much the same way as in a homopolymer. The inventors theorize, although the invention is in no way dependent on the correctness of the theory, that the nanocrystalline copolymers have a morphology-driven structure with short block runs of each comonomer to give a multi-block morphology, and that the different blocks are long enough to phase-separate before crystallization takes place, perhaps with the highly structured Cy groups close packing around clusters of the hydrophilic units (e.g. the highly polar units derived from acrylic acid, with their high potential for hydrogen bonding of the carboxylic acid group hydrogen atoms). In addition, the polymer crystal morphology may be significantly influenced by the polymer viscosity as crystallization takes place.

The SSP polymers of this invention often have molecular weights of 2,000 to 100,000, preferably 2,000 to 50,000, more preferably 3,000 to 25,000 and most preferably 3,000 to 12,000. The preferred nanocrystalline polymers generally have molecular weights less than 10,000, since higher molecular weights tend to result in increased opacity and haziness when observed in a small hardened film, indicating the presence of crystals large enough to refract light. Annealing the polymers at a temperature around Tₒ results in an increase of the opacity of the nanocrystalline polymers, presumably because the size of the crystalline domains increases during the annealing process, thereby highlighting the influence of viscosity in allowing the crystallizing side chains to co-crystallize.

The SSP Examples below indicate how variation of the different repeating units and the molecular weight influences the presence or absence of nanocrystallinity in the polymer.

### Uses of the SSPs

SSP polymers are suitable for the following exemplary uses.

### 1) Use as a Thickener, Particularly in Personal Care Compositions

SSP polymers can thicken a water phase to form stable gels and emulsions when mixed with oils including simple vegetable oils, like canola oil to make a water-in-oil emulsion or oil-in-water emulsion. Depending on the composition of the SSP polymer, it may

(a) be a good thickener of water-based compositions, in that it forms a gel in water as well as forming a stable emulsion under acidic conditions (generally SSP polymers with a molar ratio of repeating units of formula (1) to repeat units of formula (2zphil) of about 4;
(b) form a gel in water as well as stabilize emulsions under acidic conditions (generally SSP polymers with a wider range of a composition); and
(c) under acidic conditions, form a gel in water as well as stabilizing emulsions (SSP polymers generally).

Normally, one benefits by using polar comonomers to provide water phase solubility with the molecular weight of the polymer providing the thickening mechanism. In this case the thickening is a result of the intermolecular and intramolecular interaction of the long side chain groups on the polymer which overlap forming crystalline domains or associating domains and providing an associative thickening effect for the water-in-oil or oil-in-water emulsion. Because this effect is not dependent on pH, this thickening can occur at low pH, below the normal pH of neutralization (pH=6 to 7). Thus, the unneutralized SSP polymers offer an interesting alternative for thickening skin care compositions having a pH close to the pH of natural human skin (4-6), which normally requires a cationic thickener. Skin care formulations thickened by these polymers are stable once formulated and have soft and unusual texture when applied to dry skin.

After neutralization in a pH=7 or greater solution, the strong affinity of the highly polar acid groups will cause initial swelling and disruption of the crystalline regions. As a result the solutions will become amorphous and non-crystalline and still with the high concentration of acid groups retain some thickening quality similar to low molecular weight polyacrylic acid suspending agents and solutions, but such thickening is less than at the lower pH.

### 2. Use as a Hair Styling Additive

It is known to use a conventional SCC polymer as an ingredient of a hairstyling formulation. The SSP polymers, in particular the nanocrystalline polymers, can be used in the same way, and can give advantageous results. For example, the styled hair can have a shinier appearance, and the styling formulation can have better adhesion, allowing the hair to remain shiny and brilliant and non-flaking. Moreover, when the SSP polymer has a high acid content, it can be readily washed out of the hair.

### EXAMPLES

The patents and patent publications referred to above contain detailed examples of the preparation and testing of CYC carriers and pharmaceutical formulations containing CYC carriers. Those examples can be repeated replacing the drugs used in those examples by cosmetic materials and making corresponding modifications, to illustrate release compositions containing CYC carriers and cosmetic materials.

Table COS below summarizes examples in which CYC carriers based on PLGA polymers, and comparable polymers which are not CYC carriers, are tested to determine their ability to thicken different oils. Examples 1, 4, 12, 18, 21, 25, 29 and 30 are comparative Examples. In the Table, the polymers in Examples 1-28 contain units derived from lactic acid and glycolic acid in the molar ratio to each other shown in the column “L/G mol”, the units being derived from lactide and glycolide in Examples 7-11 and 16-17 and from lactic acid and glycolic acid in the other Examples. The polymers used in Examples 2, 3, 5-11, 13-17, 19-20, 22-24, and 26-28 contain Cy moieties in the percentage amount by weight, based on weight of the polymer, shown in the column “Cy % wt”, the Cy moieties being behenyl groups. The polymers are prepared using known techniques as shown for example in application Ser. No. 12/315,876, and had molecular weights of less than 6000. The polymers used in Examples 29 and 30, which are comparative examples were homopolymers of stearyl acrylate and behenyl acrylate, respectively, and had molecular weights of more than 50,000.

The oils used in the tests were mineral oil (white, heavy mineral oil from Aldrich), isopropyl palmitate (IPP) from Stepan, Neobee 1053, which is a mixture of alkyl glycerides from Stepan, Finsolve TN, which is C 12-15 alkyl benzoate, from Finetex, and castor oil from Alnoroil Company Incorporated.

The procedure used was to mix the oil and the polymer (weight ratio 90/10) at a temperature of 70-80° C. with agitation for at least 15 minutes to dissolve the polymer in the oil, followed by cooling at room temperature for at least 16 hours. The resulting product was assessed for solubility of the polymer in the oil and the ability of the polymer to thicken the oil. The solubility results are shown in the column “sol”, in which 1 denotes a clear solution, 2 denotes a slightly hazy solution, 3 denotes that the polymer is not soluble or only partially soluble. The ability of the polymer to thicken the oil is shown in the column “gel”, in which a indicates the formation of a gel which is soluble in the oil, b indicates the formation of a gel, which is partially soluble in the oil, c indicates the formation of a gel with small amounts of free oil, and d indicates that no gel was formed. In the Table, the indications a, b and c are accompanied by the results of viscosity measurements carried out on the gels at 25° C., using a Brookfield DV-I+ viscometer equipped with cone and plate spindle CP51 at 2.5 rpm, the data points being taken at 30, 60, 120 and 240 seconds and reported in cps as an average of these four data points.

TABLE COS 1

| | | | | Mineral | | Isopropyl. | | Neobee | | Finsolve | | | |
|---|---|---|---|---|---|---|---|---|---|---|---|---|---|
| Ex # | Polymer | L/G | Cy % | oil | | palmitate | | 1053 | | TN | | castor oil | |
| PC | ID | mol | wt | sol | gel | sol | gel | sol | gel | sol | gel | sol | gel |
| 1 | 341-18-0 | 75/25 | 0 | 3 | d | 3 | d | 3 | d | 3 | d | 3 | d |
| 2 | 341-18-1 | 75/25 | 32 | 2 | d | 1 | d | 1 | d | 1 | 8764^{a} | 1 | 2047^{c} |
| 3 | 341-18-3 | 75/25 | 11 | 3 | d | 3 | d | 3 | d | 3 | d | 3 | d |
| 4 | 341-1-0 | 50/50 | 0 | 3 | d | 3 | d | 3 | d | 3 | d | 3 | d |
| 5 | 341-1-1 | 50/50 | 33 | 3 | d | 2 | d | 3 | d | 1 | d | 1 | d |
| 6 | 341-1-3 | 50/50 | 11 | 3 | d | 3 | d | 3 | d | 3 | d | 3 | d |
| 7 | 336-176-1 | 50/50 | 33 | 3 | 25698^{c} | 1 | 16507^{c} | 3 | 3566^{c} | 1 | d | 1 | 1351^{c} |
| 8 | 336-176-3 | 50/50 | 11 | 3 | d | 3 | d | 3 | d | 3 | d | 3 | d |
| 9 | 342-36-1 | 75/25 | 33 | 2 | 1433^{c} | 1 | d | 1 | d | 1 | d | 1 | 3563^{c} |
| 10 | 342-36-3 | 75/25 | 11 | 3 | d | 3 | d | 3 | d | 3 | d | 3 | d |
| 11 | 342-150-2 | 100/0 | 20 | 2 | d | 2 | 4009^{c} | 2 | d | 2 | d | 2 | d |
| 12 | 346-110 | 50/50 | 0 | 3 | d | 3 | d | 3 | d | 3 | d | 3 | d |
| 13 | 346-115 | 50/50 | 10 | 3 | d | 3 | d | 3 | d | 3 | d | 3 | d |
| 14 | 346-133 | 50/50 | 30 | 3 | d | 1 | d | 1 | d | 1 | d | 3 | d |
| 15 | 346-19 | 75/25 | 30 | 3 | d | 1 | 1515^{a} | 3 | 860^{a} | 1 | d | 3 | 983^{b} |
| 16 | 346-137W | 75/25 | 30 | 3 | d | 1 | 3072^{a} | 3 | 327^{c} | 1 | 2416^{a} | 3 | 1638^{b} |
| 17 | 346-147W | 50/50 | 20 | 3 | d | 1 | d | 3 | d | 3 | d | 3 | d |
| 18 | 346-111 | 50/50 | 0 | 3 | d | 3 | d | 3 | d | 3 | d | 3 | 901^{c} |
| 19 | 346-32 | 50/50 | 10 | 3 | 10238^{b} | 3 | 2621^{c} | 3 | 491^{c} | 3 | 1064b | 3 | 1597^{b} |
| 20 | 346-56 | 50/50 | 30 | 1 | 6635^{a} | 1 | 163^{a} | 3 | 368^{c} | 3 | 47640^{b} | 3 | 1310^{c} |
| 21 | 342-18-1 | 50/50 | 0 | 3 | d | 3 | d | 3 | d | 3 | d | 3 | d |
| 22 | 342-69-1 | 50/50 | 30 | 3 | 532^{c} | 1 | 778^{c} | 1 | d | 1 | d | 1 | d |
| 23 | 342-69-2 | 50/50 | 20 | 3 | d | 3 | d | 3 | d | 3 | d | 3 | d |
| 24 | 342-69-3 | 50/50 | 10 | 3 | d | 3 | d | 3 | d | 3 | d | 3 | d |
| 25 | 342-71 | 75/25 | 0 | 3 | d | 3 | d | 3 | d | 3 | d | 3 | d |
| 26 | 342-74-1 | 75/25 | 30 | 3 | d | 1 | 573^{c} | 3 | d | 1 | d | 3 | 2416^{c} |
| 27 | 342-74-2 | 75/25 | 20 | 3 | d | 3 | 1269^{c} | 3 | d | 3 | d | 3 | d |
| 28 | 342-74-3 | 75/25 | 10 | 3 | d | 3 | d | 3 | d | 3 | d | 3 | d |
| 29 | 13-1 | N/A | N/A | 1 | 58610^{a} | 1 | 44192^{a} | 1 | 66232^{a} | 1 | 36162^{a} | 2 | 1597^{c} |
| 30 | 13-6 | N/A | N/A | 1 | 69135^{a} | 1 | 49885^{a} | 1 | 71470^{a} | 1 | 67352^{a} | 2 | 8439^{c} |

### Examples PC 31-35

Examples PC 31-35 make use of a 6ECC-PLGA polymer prepared by the following procedures A and B described below.

### Procedure A—Preparation of Prepolymer

Glycolic acid (99%, 336.2 g), lactic acid solution (85+% in water, 463.8 g), D-Sorbitol (97%, 31.6 g) and toluene (tech grade, 500 gram) were weighed into a 2000 ml reaction vessel equipped with a mechanical stirrer and condenser.

The loaded reaction vessel was heated in a 150° C. oil bath and refluxed under nitrogen. After 24 hr of refluxing, about 100 gram of water was collected. The reaction was continued under reduced pressure in a 150° C. bath for at least 16 hrs. A clear pale yellow viscous liquid was produced and removed from the reactor while it was still hot. After cooling to room temperature, 852 gram of clear colorless solid was obtained and labeled 342-18-1. Thermal analysis by DSC showed To at 61.38° C. and Tp at 63.76° C. The polymer exhibited Mw of 2525 and Mn of 1155 in averaging 2 runs by GPC using polystyrene as standard.

### Procedure B—Preparation of 6ECC-PLGA

Prepolymer 342-18-1 (81.5 g), behenic acid (35 g, recrystallized twice from ethylacetate) and a catalytic amount of tin-2ethylhexanoate (0.11 gram) were combined in a 1000 mL reaction vessel with 350 gram of toluene. The reaction was refluxed in a 150° C. bath for 3 days under nitrogen blanket. Toluene was removed under reduced pressure over 4 hours followed by continued heating under nitrogen at 150° C. for 4 days until completely reacted. Reaction progress was monitored by DSC. The completion of the reaction on the DSC was monitored by the disappearance of the peak between 75 to 80° C. due to behenic acid. The final product of 104 gram was collected as a white solid and labeled 342-69-1. Thermal analysis by DSC showed To at 61.38° C. and Tp at 63.76° C. and Tg at 31.8° C. The polymer exhibited Mw of 4423 and Mn of 1605 in averaging 2 runs by GPC using polystyrene as standard.

### Example PC31

A composition suitable for a lipstick is prepared by mixing the following ingredients at 70° C., followed by cooling to ambient temperature. C18 functional polymer (6 g), 6ECC-PLGA (6 g), parleam oil (8 g), pigment paste (1 g) and hydrogenated isoparafin (79 g),

### Example PC32

A composition suitable for a mascara is prepared by the following steps. In step (1), 6ECC-PLGA (23.3 g), polybutene (11.7 g), aminopropanediol (23.3 g), stearic acid (5.8 g), pigments (8 g), and hydroxyethyl cellulose (0.9 g) were combined and heated to 70° C. with stirring. In step (2), Gum Arabic (3.45 g), triethanolamine (2.4 g), preserving agents and water to 100 g were added, thus forming an emulsion that was stable when cooled.

### Example PC33

A composition suitable for a skin care cream is prepared by the following steps. In step (1), an oil phase was prepared by combining at 70° C. 6ECC-PLGA (1.2 g), parleam oil (0.8 g), isohexadecane (15 g), silicone oil (10 g), c etyldimethicone polyol (Abil EM90) (1.5 g) and polyglycerylisostearate (Isolan G134) (0.5 g). In step (2), a water phase was prepared by combining at 70° C. glycerol (3 g), magnesium sulfate (0.7 g). In step (3), the aqueous phase was dispersed in the oil phase with agitation, and the dispersion was cooled to 40° C., at which time the starch powder (3 g) was added, followed by further cooling.

### Example PC34

A composition suitable for a complexion cream is prepared by the following steps. In step (1), an oil phase was prepared by combining at 70° C. 6ECC-PLGA (1.2 g), parleam oil (0.8 g), isohexadecane (10 g), volatile silicone oil (7 g), isononyl isononanoate (5 g), sweet almond oil (0.5 g), polyglycerylisostearate (Isolan G134) (0.5 g), a mixture of isostearyl glucoside and isostearyl alcohol (Montanov WO18 Seppic) (5 g) and coated iron oxides (brown, yellow, black) (0.5 g). In step (2), the water phase was prepared by combining at 70° C. glycerol (3 g), magnesium sulfate (0.7 g), preserving agents and water to 100 g. In step (3), the aqueous phase was dispersed in the oil phase with agitation, and the dispersion was cooled to 50° C. ° C., at which time a mixture of polyamide powder (6 g), polyethylene powder (4 g) and fragrances (0.5 g) was added, followed by further cooling.

### Example PC35

A composition suitable for a make-up removal stick is prepared by the following steps. In step (1), an oil phase was prepared by combining at 80° C. 6ECC-PLGA (4.8 g), parleam oil (3.2 g), isohexadecane (15 g), cetyldimethicone polyglyceryl isostearate/hexyl laurate (Abil WE09) (5 g), isopropyl palmitate (45 g) and nylon 12 (Orgasol 2002) (1.5 g). In step (2), a water phase was prepared by combining at 80° C. glycerol (5 g), magnesium sulfate (0.7 g), preserving agents and distilled water to 100 g. In step (3), the aqueous phase was dispersed in the oil phase with agitation, followed by cooling.

The following Examples illustrate the preparation of SSP polymers and the results of mixing them with water or with water and canola oil.

### SSP EXAMPLES

### Example PC 36

### Preparation of SSP polymers

Table SSP 1 below summarizes the preparation of SSP polymers by solution polymerization of the acrylic monomers and amounts thereof identified in the table. The polymerization was carried out in IPA at 80° C. for 3 hours under nitrogen with 0.1% AIBN. In some cases, BMP (6%) was used to control the molecular weight below 5000 Daltons. IPA was removed under reduced pressure at elevated temperature. At the end of the reduced pressure stage, the internal temperature reached 120-130° C. In general, 0.5 g of Trigonox was added to lower the residual monomer. The reaction was continued at 120-130° C. for at least 1 hr, followed by 1 hr under reduced pressure. However, if the viscosity was too high to stir during or after IPA removal then samples were taken “as is” and put under reduced pressure at 90° C. overnight to remove the IPA.

TABLE SSP 1

| | | | | | | |
|---|---|---|---|---|---|---|
| C18A | C22A | AA | C18A/C22A/AA | Thermal transition temperature (° C.) | Average | Average |

| | | | | | | | |
|---|---|---|---|---|---|---|---|
| ID | wt % | molar ratio | 1st heating | 2nd heating | 1st cooling | (Mw) | (Mn) |

Low Mw set with 6% BMP in IPA as solvent

| | | | | | | | | | |
|---|---|---|---|---|---|---|---|---|---|
| 328-57-1 | 60 | | 40 | 1/0/3 | 46.0 | 40.5 | 20.0 | 4504 | 2983 |
| 328-57-2 | 52.9 | | 47.1 | 1/0/4 | 41.8 | 25.3 | 13.4 | 3530 | 2658 |
| 328-57-3 | 47.4 | | 52.6 | 1/0/5 | 42.5 | 41.0 | 12.4 | 3209 | 2445 |
| 328-57-4 | | 63.8 | 36.2 | 0/1/3 | 65.0 | 63.1 | 53.2 | 2774 | 1931 |
| 328-57-5 | | 56.9 | 43.1 | 0/1/4 | 66.7 | 63.4 | 43.8 | 2976 | 2140 |
| 328-57-6 | | 51.4 | 48.6 | 0/1/5 | 65.7 | 63.2 | 39.0 | 3083 | 2229 |
| 328-57-7 | 16.8 | 45.9 | 37.3 | 0.3/0.7/3 | 61.9 | 58.9 | 44.2 | 2947 | 1947 |
| 328-57-8 | 14.9 | 40.8 | 44.2 | 0.3/0.7/4 | 60.7 | 57.2 | 36.6 | 12645 | 7136 |
| 328-57-9 | 13.4 | 36.8 | 49.8 | 0.3/0.7/5 | 58.1 | 43.0 | 29.9 | 3075 | 2051 |

High Mw set with no BMP in IPA as solvent

| | | | | | | | | | |
|---|---|---|---|---|---|---|---|---|---|
| 328-57-10 | 60 | | 40 | 1/0/3 | 40.7 | 22.6 | 13.9 | 12320 | 5544 |
| 328-57-11 | 52.9 | | 47.1 | 1/0/4 | 47.5 | 25.2 | 16.3 | 12400 | 6774 |
| 328-57-12 | 47.4 | | 52.6 | 1/0/5 | 34.2 | 22.4 | 8.6 | 14825 | 7623 |
| 328-57-13 | | 63.8 | 36.2 | 0/1/3 | 70.8 | 64.3 | 43.5 | 11915 | 3986 |
| 328-57-14 | | 56.9 | 43.1 | 0/1/4 | 67.5 | 56.1 | 40.7 | 12725 | 5464 |
| 328-57-15 | | 51.4 | 48.6 | 0/1/5 | 67.6 | 51.0 | 39.1 | 13185 | 6340 |
| 328-57-16 | 16.8 | 45.9 | 37.3 | 0.3/0.7/3 | 53.2 | 52.1 | 34.8 | 12015 | 5049 |
| 328-57-17 | 14.9 | 40.8 | 44.2 | 0.3/0.7/4 | 58.5 | 47.6 | 40.4 | 11630 | 4973 |
| 328-57-18 | 13.4 | 36.8 | 49.8 | 0.3/0.7/5 | 57.1 | 54.3 | 32.6 | 3109 | 2160 |

### Example PC 37

### Solubility of 5% Polymer in Water

Table SSP 2 below shows the results of mixing 0.5 gram of the polymers prepared in Table SSP 1 and 9.5 gram DI water, heating the mixture to 70 to 80° C. for about 1 hr, or shaking the hot mixture, and maintaining the mixture at 70-80° C. for 30-60 minutes. The hot mixture was hand shaken to mix. The resulting mixture was held at 70-80° C. for 30-60 min, and its solubility and clarity is reported in Table SSP 2. The mixture was transferred to a 25° C. oven overnight, and table SSP 2 records whether or not a gel formed.

TABLE SSP 2

| | | SCC/COOH | Intelimer | DI water | Uniformly dispersible | |
|---|---|---|---|---|---|---|
| Gel ID | Interlimer | molar ratio | g | g | in water at 80 C.? | Gel at 25 C.? |
| 328-79-1 | 328-57-1 | 1/3 | 0.50 | 9.5 | yes | yes |
| 328-79-2 | 328-57-2 | 1/4 | 0.50 | 9.5 | yes | yes |
| 328-79-3 | 328-57-3 | 1/5 | 0.50 | 9.5 | yes | no |
| 328-79-4 | 328-57-4 | 1/3 | 0.50 | 9.5 | no | no |
| 328-79-5 | 328-57-5 | 1/4 | 0.50 | 9.5 | yes | yes |
| 328-79-6 | 328-57-6 | 1/5 | 0.50 | 9.5 | yes | no |
| 328-79-7 | 328-57-7 | 1/3 | 0.50 | 9.5 | almost yes | yes |
| 328-79-8 | 328-57-8 | 1/4 | 0.50 | 9.5 | yes | yes |
| 328-79-9 | 328-57-9 | 1/5 | 0.50 | 9.5 | yes | no |
| 328-79-10 | 328-57-10 | 1/3 | 0.50 | 9.5 | no | no |
| 328-79-11 | 328-57-11 | 1/4 | 0.50 | 9.5 | no | no |
| 328-79-12 | 328-57-12 | 1/5 | 0.50 | 9.5 | no | no |
| 328-79-13 | 328-57-13 | 1/3 | 0.50 | 9.5 | no | no |
| 328-79-14 | 328-57-14 | 1/4 | 0.50 | 9.5 | yes | no |
| 328-79-15 | 328-57-15 | 1/5 | 0.50 | 9.5 | no | no |
| 328-79-16 | 328-57-16 | 1/3 | 0.50 | 9.5 | no | no |
| 328-79-17 | 328-57-17 | 1/4 | 0.50 | 9.5 | yes | no |
| 328-79-18 | 328-57-18 | 1/5 | 0.50 | 9.5 | no | no |

### Example PC 38

### Solubility of 10% Polymer in Water

The procedure of 2 was repeated, but using a mixture of 1.0 gram polymer and 9.0 gram DI water. The results are recorded in Table SSP 3 below.

TABLE SSP 3

| | | SCC/COOH | Intelimer | DI water | Uniformly dispersible | |
|---|---|---|---|---|---|---|
| Gel ID | Intelimer | molar ratio | g | g | in water at 80 C.? | Gel at 25 C.? |
| 328-80-1 | 328-57-1 | 1/3 | 1.00 | 9.00 | almost yes | yes |
| 328-80-2 | 328-57-2 | 1/4 | 1.00 | 9.00 | yes | yes |
| 328-80-3 | 328-57-3 | 1/5 | 1.00 | 9.00 | yes | yes |
| 328-80-4 | 328-57-4 | 1/3 | 1.00 | 9.00 | no | no |
| 328-80-5 | 328-57-5 | 1/4 | 1.00 | 9.00 | yes | yes |
| 328-80-6 | 328-57-6 | 1/5 | 1.00 | 9.00 | yes | yes |
| 328-80-7 | 328-57-7 | 1/3 | 1.00 | 9.00 | no | no |
| 328-80-8 | 328-57-8 | 1/4 | 1.00 | 9.00 | yes | yes |
| 328-80-9 | 328-57-9 | 1/5 | 1.00 | 9.00 | yes | yes |
| 328-80-10 | 328-57-10 | 1/3 | 1.00 | 9.00 | phase separated | no |
| 328-80-11 | 328-57-11 | 1/4 | 1.00 | 9.00 | phase separated | no |
| 328-80-12 | 328-57-12 | 1/5 | 1.00 | 9.00 | phase separated | no |
| 328-80-13 | 328-57-13 | 1/3 | 1.00 | 9.00 | phase separated | no |
| 328-80-14 | 328-57-14 | 1/4 | 1.00 | 9.00 | no | no |
| 328-80-15 | 328-57-15 | 1/5 | 1.00 | 9.00 | phase separated | no |
| 328-80-16 | 328-57-16 | 1/3 | 1.00 | 9.00 | phase separated | no |
| 328-80-17 | 328-57-17 | 1/4 | 1.00 | 9.00 | no | no |
| 328-80-18 | 328-57-18 | 1/5 | 1.00 | 9.00 | phase separated | no |

### Example PC 39

### Emulsions

Canola oil (4.2 gram) and a solution of each of the polymers in Table SSP 1 in DI water (0.51 gram polymer in 10 gram DI water) were separately heated to 70-80° C. for 30 min. The warm Canola oil was poured into the warm aqueous polymer solution and immediately mixed with a homogenizer for 1 min while cooling in an ice bath. The resulting emulsion was kept at 25° C. overnight. Table SSP 4 reports the results.

TABLE SSP4
Emulsion formulation

| | | SCC/COOH | wt | DI water | Canola oil | Uniformly dispersible | stable emulsion at 25 C. | flowable |
|---|---|---|---|---|---|---|---|---|
| ID | Intelimer | molar ratio | g | g | g | in water at 80 C.? | after 2 days? | at 25 C.? |
| 328-81-1 | 328-57-1 | 1/3 | 0.5145 | 9.996 | 4.190 | mostly yes | yes | no |
| 328-81-2 | 328-57-2 | 1/4 | 0.5145 | 9.996 | 4.190 | yes | yes | no |
| 328-81-3 | 328-57-3 | 1/5 | 0.5145 | 9.996 | 4.190 | yes | no | no |
| 328-81-4 | 328-57-4 | 1/3 | 0.5145 | 9.996 | 4.190 | mostly yes | no | yes |
| 328-81-5 | 328-57-5 | 1/4 | 0.5145 | 9.996 | 4.190 | yes | yes | no |
| 328-81-6 | 328-57-6 | 1/5 | 0.5145 | 9.996 | 4.190 | yes | no | yes |
| 328-81-7 | 328-57-7 | 1/3 | 0.5145 | 9.996 | 4.190 | mostly yes | no | no |
| 328-81-8 | 328-57-8 | 1/4 | 0.5145 | 9.996 | 4.190 | yes | yes | no |
| 328-81-9 | 328-57-9 | 1/5 | 0.5145 | 9.996 | 4.190 | yes | no | yes |
| 328-81-10 | 328-57-10 | 1/3 | 0.5145 | 9.996 | 4.190 | phase separated | yes | yes |
| 328-81-11 | 328-57-11 | 1/4 | 0.5145 | 9.996 | 4.190 | no | no | yes |
| 328-81-12 | 328-57-12 | 1/5 | 0.5145 | 9.996 | 4.190 | phase separated | no | yes |
| 328-81-13 | 328-57-13 | 1/3 | 0.5145 | 9.996 | 4.190 | no | no | no |
| 328-81-14 | 328-57-14 | 1/4 | 0.5145 | 9.996 | 4.190 | no | yes | yes |
| 328-81-15 | 328-57-15 | 1/5 | 0.5145 | 9.996 | 4.190 | phase separated | no | yes |
| 328-81-16 | 328-57-16 | 1/3 | 0.5145 | 9.996 | 4.190 | phase separated | yes | yes |
| 328-81-17 | 328-57-17 | 1/4 | 0.5145 | 9.996 | 4.190 | no | yes | yes |
| 328-81-18 | 328-57-18 | 1/5 | 0.5145 | 9.996 | 4.190 | phase separated | no | yes |

### Example PC 40

### Additional Polymers

Three additional polymers were prepared using a method similar to that in Example PC 36.

(A) A copolymer prepared from C18 A/AA/C1A, molar ratio 1/3.5/0.5, exhibited significant opacity similar to standard SCC polymer. On the first heat, it had a melting temperature of about 46° C. and a heat of fusion of about 43 J/g. On the second heat, it had a Tp of about 43° C. and a heat of fusion of about 37 J/g. It had a much lower viscosity than the C18A/AA, molar ratio 1/4, polymer. This result shows the influence of polymer viscosity in allowing the crystallizing side chains to align and crystallize.
(B) A copolymer prepared from C18A and acrylamide, molar ratio 1/4, is a clear crystalline polymer. On the first heat, it had a melting temperature of about 23° C. and a heat of fusion of about 19 J/g. On the second heat, it had a Tp of about 22° C. and a heat of fusion of about 20 J/g.
(C) A copolymer of C18A/AA (molar ratio 1/4)/MCR-M17 containing 5 weight percent MCR-M17 is a translucent polymer at room temperature. On the first heat, it had a melting temperature of about 39° C. and a heat of fusion of about 30 J/g. On the second heat, it had a Tp of about 38° C. and a heat of fusion of about 27 J/g.

1. A personal care composition which comprises a CYC carrier as hereinbefore defined, and a cosmetic material as hereinbefore defined, the composition having at least one of the following characteristics:
(1) the CYC carrier comprises an ECC polymer as hereinbefore defined,
(2) the CYC carrier comprises a CYC compound as hereinbefore defined,
(3) the CYC carrier comprises an SSP polymer as hereinbefore defined,
(4) the CYC carrier comprises a CYC assembly as hereinbefore defined,
(5) the CYC carrier is bioerodable, and
(6) the cosmetic material comprises a Cy moiety as hereinbefore defined.

2. A personal care composition according to claim 1, which is selected from the group consisting of deodorants, anti-perspirants, lipsticks, makeups, lotions, creams, oil-in-water emulsions, water-in-oil emulsions, hair care compositions, color care compositions, skin care compositions, sun care compositions, hair styling compositions, hair restyling compositions, shampoos, conditioners, nail varnishes, gels, oils, creams, hand creams, night renewal creams, body milks, body lotions, light facial creams, protective day creams, liquid moisturizing emulsions, and products designed to remove makeup/personal care products.

3. A personal care composition which comprises a CYC carrier as hereinbefore defined, a cosmetic material as hereinbefore defined, and an additive which self assembles with the CYC carrier to increase the Tp of the composition by at least 1° C., preferably at least 3° C., and/or to increase the heat of fusion of the composition by at least 2, preferably at least 3, J/g.

4. A method of changing the appearance, feel, hygiene or smell of a human body which comprises applying to the body a personal care composition as defined in claim 1.

5. A method of thickening an oil which comprises mixing the oil with a CYC carrier as hereinbefore defined, the CYC carrier having at least one of the following characteristics:—
(1) it comprises an ECC polymer as hereinbefore defined,
(2) it comprises a CYC compound as hereinbefore defined,
(3) it comprises an SSP polymer as hereinbefore defined,
(4) it comprises a CYC assembly as hereinbefore defined,
(5) it comprises a CYC compound as hereinbefore defined,
(6) it is bioerodable.
